(19) United States
(12) Patent Application Publication
(10) Pub. No.: US 2018/0308569 A1
(43) Pub. Date: Oct. 25, 2018

(54) SYSTEM OR METHOD FOR ENGAGING PATIENTS, COORDINATING CARE, PHARMACOVIGILANCE, ANALYSIS OR MAXIMIZING SAFETY OR CLINICAL OUTCOMES

(71) Applicant: S Eric Luellen, Belmont, MA (US)

(72) Inventor: S Eric Luellen, Belmont, MA (US)

(21) Appl. No.: 15/496,950

(22) Filed: Apr. 25, 2017

(51) Int. Cl. G16H 20/10; G16H 10/60; G16H 50/20

(52) U.S. Cl. CPC G16H 20/10; G16H 10/20; G16H 50/20; G16H 10/60

(57) ABSTRACT

The invention provides a Platform or Application, via numerous systems and methods, involving software, code, pseudo-code, databases, schemas, algorithms, inference engines, and user interfaces including but not limited to Complex Event Processing (CEP) artificial intelligence, for pharmacotherapy management and evaluation (specifically managing medication therapy regimens), maximizing patient safety and Medication efficacy, coordinating care among disparate Providers, improving clinical outcomes, regulatory reporting, and research, learning, or knowledge discovery via analysis. More specifically, the invention is comprised of a mobile and digital health intervention platform with various modules addressing health challenges related to medication non-adherence behaviors, predicting and preventing medication non-adherence, identifying and overcoming reasons for non-adherence, identifying risks or preventing adverse drug interactions (ADR) (e.g., between medications (polypharmacy), environments, age or consumables), and/or adverse events (AE), automatically tracking and reporting adverse events (AE), coordinating care amongst a diversified healthcare treatment team (medical concordance), or conducting Analysis, research, knowledge discovery or learning regarding the data (events). Users include Patients, caregivers, health care Providers, Pharmaceutical Manufacturers, Insurance Carriers, National Health Services, Payers, or other interested parties.

### BACKGROUND

### Field of Disclosure

The invention is in the general field of health care, patient support, and medication therapy management. More specifically, it is in the fields of medication adherence, pharmacovigilance, and coordination of care

### Description of the Related Art

Three preventable health-management problems cost humanity over 420,000 lives, and over $664 billion every year: (1) medication non-adherence; (2) pharmacovigilance; and, (3) coordination of care. The overall goals of solutions are saving billions of dollars by preventing health complications, and improving clinical outcomes. The systems and methods disclosed herein solve a series of problems in these three areas; here's what they are and how.

In medication non-adherence, over 250,000 lives and $594 billion in lost revenue and preventable medical expenses are spent each hear because patients don't take their medications as prescribed. Two of the primary reasons for that are forgetfulness and complexity of a regimen (many drugs). While apps exist to remind patients to take a single drug (like an alarm clock), there are no known platforms that track the entire regimen with metadata and allow patients to custom-set reminders based on their communication preferences and priority of the medication, coordinate across a health care treatment team, prevent medication interactions, and monitor adverse reactions in real time. Our systems and methods, among other things, allow for all this such that a patient with 20 drugs who prefers high-priority reminders via phone call, mid-priority by SMS text, and low-priority by direct-messaging apps (Facebook Messenger, WhatsApp, etc.) or e-mail is all enabled. Moreover, providers (physicians and pharmacists) today don't know which patients take which medications and how often. Our platform compiles and reports analytics to Payers and Providers on their patients such that they know in near-real time, and prior to appointments to discuss, which patients are adhering, how much, when, etc. In essence, the Platform monitors many Patient-centric Medication regimen events, prevents negative events, prioritizes information patterns to identify problems, and alerts members' of a Patient's health care treatment team when the occur. In collecting large quantities of Patient behavior data, the Platform also creates a long-term opportunity for data mining, analysis, and research and to provide these results and knowledge via Big Data as a Service (BDaaS), also known as Infonomics.

Pharmacovigilance, or drug safety, costs an estimated 185,000 lives per year and up to $100 billion in preventable medical expenses, either from drug-drug interactions, drug-food interactions, or adverse events that may be related to personalized medical issues. Our platform solves these problems by screening for drug-drug interactions, and drug-food interactions, as soon as the regimen is entered into the system.

Adverse events are when a patient reacts unexpectedly, often badly, to a medication. This is a special regulatory concern of the FDA that is costly for pharmaceutical manufactures to comply with reporting, much less prevent. Our platform has taken the FDA adverse-event reporting form and digitized to be used on smart devices—wherein the patient answers a few questions, hits send, and it's automatically transmitted to the patient's treatment team, pharmacists, medical insurers, drug manufacturers, and the FDA.

A secondary issue that encompasses both medication non-adherence and pharmacovigilance is the absence of information in the hands of people who need it when they need it. For example, a next-of-kin or physician may want or need to know when a patient has failed to take their heart medication, or is having a particular type of adverse reaction, or an interaction of a certain type of severity is flagged. Today, there is no known system to analyze these events in real time and act on them. The systems and methods of our platform enable complex event process (CEP), a form of artificial intelligence (AI) and operational intelligence (OD, that tracks, records, and acts on these events, getting actionable intelligence to the people that need to know, when they need to know, and in the communication medium they prefer (e.g., call, email, SMS Text, direct message, etc.).

Coordination of care largely relates to medical concordance, which is the industry term for prescriptions from multiple physicians and/or pharmacies such that the treatment team doesn't know about each other, which compound over time. The systems and methods of our platform allow the patient and their entire treatment team to work from one list that is accessible to everyone. The entire treatment team knows what the patient is taking, in what dosage, when it starts and stops, and about any interactions or adverse events. Moreover, medical concordance via our platform's systems and methods makes it much more difficult for patients to commit prescription drug fraud by getting multiple prescriptions from multiple physicians or pharmacies.

According to a Capgemini study in 2015, the US mortality related medication non-adherence is approximately 220,000 lives per year, with a financial loss of approximately $300 billion annually. Similarly, in the United Kingdom, a study commissioned by Omnicell determined mortality related to medication non-adherence at 200,000 deaths per year, and financial losses of approximately 500 million pounds annually.

How and why? Statistics tell the story. According to Anthem, a major American insurance carrier, 137 million Americans now take a prescription drug to manage a chronic condition, which it predicts will increase to 157 million by 2020. Approximately 10.6% of those patients are taking more than five drugs simultaneously (referred to as polypharmacy), and for patients over 45 years of age, 29% are taking more than five drugs simultaneously. Separate surveys in the US (2006) and UK (2015), independently found that 75% of patients failed taking medications as directed. According to a 2005 study published in the New England Journal of Medicine, 33% of hospitalizations in the US are now related to medication non-adherence. Anthem also identified 700,000 dosing-related emergencies per year. To put those statistics into global perspective, the US has only 4.6% (321 million) of the global population (7.28 billion), and the UK has only 0.009% (64 million) of the world's population (7.28 billion).

Pharmacovigilance is defined by the World Health Organization (WHO) as: “the science and activities relating to the detection, assessment, understanding and prevention of adverse effects or any other drug-related problem.” Alternatively, pharmacovigilance is the science of collecting, monitoring, researching, assessing, and evaluating information from healthcare providers and patients on the adverse effects of medications with a view towards identifying hazards associated with the medications and preventing harm to patients. According to the Center for Education Research on Therapeutics, the US financial costs associated with adverse drug reactions (ADR) or adverse events (AE) is $136 billion annually. Patients with ADRs make up 20% of injuries or deaths in hospital patients, and their hospital stays are 200% of the length, cost, and mortality compared to control groups. They estimate over 2 million serious ADRs occur in the United States each year, contributing approximately 100,000 deaths. Of the estimated 2 million serious ADRs that occur in the US each year, 350,000 are in nursing homes. According to a 2008 study published in the journal Oncology, costs associated with ADRs in polypharmacy cases—unidentified drug conflicts by those on more than four medication regimens simultaneously—accounts for $76 billion in avoidable costs and a disproportionate percentage of injuries and fatalities.

Preventable care coordination challenges round out the healthcare problem trifecta. According to a 2012 study published in Health Affairs, preventable care coordination issues are costing $24-40 billion per year in the United States. Moreover, according to an Institute of Medicine (IOM) 2001 study, Crossing the Quality Chasm, 20% of fee-for-use Medicare beneficiaries who are discharged from the hospital are readmitted within 30 days—an estimated 75% of those are believed to be preventable from improved care coordination—representing a savings of $12 billion per year.

Care coordination is also a problem for hospital patients are not readmitted. A 2007 literature review published in the Journal of the American Medical Association (JAMA) found that only 12-34% of doctors had a copy of the hospital discharge summary at the patients' first post-discharge visit.

In the US, over the past decade, medication management systems have been a focal point of improving health care while reducing costs. In 2003, the Medicare Modernization Act required Medicare Part D prescription drug plans to include a medication therapy management (MTM) service component; however, there were no financial incentives to doing so. Therefore, in 2010, the Patient Protection and Affordable Care Act introduced a five-star rating system providing financial incentives for MTM. Seventeen (17) of the 53 key quality measures in the Medicare Star-rating system relate to prescription drug management. Moreover, many of those elements are weighted by 150-300% making them key contributors to a provider's overall score. The US government has projected Medicare Star Rating rebates or financial incentives to exceed $2 billion in 2015. These incentives create both a regulatory-driven and financial business case for hospital corporations to invest in patient engagement systems that address MTM needs. The Centers for Medicare & Medicaid Services (CMS) can also reduce payments by 1% to hospitals whose readmission rates exceed targets—readmissions often caused or materially contributed to by medication adherence, drug interactions and/or care coordination. According to analysis performed by the Kaiser Family Foundation in 2012, approximately 2,200 hospitals forfeited a combined estimate of $280 million in Medicare payments in 2013.

The systems and methods of this this solution are at the intersection, or confluence, of numerous modern and next-generation technologies. The technologies that are converging here include: Active data streaming, Big Data, Big Data as a Service (BDaaS), Complex event processing (CEP), Cloud computing, home and mobile health monitoring, Infonomics, the Internet-of-Things (IoT), on-line analytical processing (OLAP), predicative and social analytics.

It also represents a unique ecosystem of data. While various combinations of these technologies have been employed to, for example, target advertising for consumer purchases on-line, or direct or re-direct traffic routes, no one to date has used complex event processing (CEP) specifically to: (1) create value in the velocity of pharmacotherapy data; or, (2) aggregate external events with pharmacotherapy management for analysis and action triggering. Our solution also provides continuous (versus batch) analysis of streams of data in motion as it relates to patients' pharmacotherapy treatment.

Prior art relates to hardware, such as docking trays that hold medicine (publication number U.S. Pat. No. 7,369,919 B2), wireless devices (e.g., pill boxes or lids that send wireless signals), or apps that enhance pre-existing alarms on smartphones. No prior art constitutes all or part of the comprehensive solution invented here including all Patient information for Storage or Analysis, multi-modal regimen-wide reminders, nor do they include pharmacovigilance nor coordination of care solutions. Our solution determines what data is important, when, why, and transmits it to the people that need to know when they need to know it. Virtually all prior are serves as reference, instead of processing data in a value-added way.

### SUMMARY

Embodiments of the invention provide a platform and Application, via numerous systems and methods, for pharmacotherapy management, specifically managing medication therapy regimens, maximizing patient safety, maximizing medication efficacy, coordinating care, improving clinical outcomes, regulatory reporting, or research, discovery, learning or Analysis.

More specifically, the invention is comprised of a mobile and digital health intervention platform employing Complex Event Processing (CEP) artificial intelligence via Cloud Computing and Software as a Service (SaaS) with various modules addressing health challenges related to medication adherence, predicting and preventing medication non-adherence, identifying reasons for non-adherence, identifying risks or preventing adverse interactions (ADR) between medications (polypharmacy) and/or adverse events (AE), automatically tracking and reporting adverse events (AE), coordinating care amongst a diversified healthcare treatment team, or conducting Analysis, research or learning regarding the data collected. The invention consists of systems and methods embodied on a series of modules operating independently, collectively or in any combination, and multi-step algorithms, for medication adherence, pharmacovigilance, or coordination of care.

The invention is a software application, for example running within the operating system of the User Device, in one or more Modules. The software application contains program Modules to implement the functionality described herein. The program modules are made up of code and pseudo-code, based on formal structures and schemas of data, and require flow chart algorithms and a rules-based inference engine. Systems and methods here encapsulate Patient-centric approaches implementing Web 2.0 methodologies such as direct messaging and SMS text messaging, and creating interactive care-coordination communities; the Application systematically identifies and categorizes medication non-adherence risk factors.

The network provides a communication infrastructure between the server(s) and User Devices. The network is typically the Internet; however, could be any Transmission Method or network.

Embodiments of the computer-readable storage medium store computer-executable instructions for performing the steps described herein. Embodiments of the system further comprise a processor(s) for executing the computer-executable instructions.

Users may include, but are not limited to, Patients, Patient family members, emergency contacts, Health Care Providers (e.g., physicians, psychiatrists, psychologists, pharmacists, or any of their authorized employees), Insurance Carriers or Payers, nursing home or ambulatory care centers or their agents or employees, or pharmaceutical manufacturers or their agents or employees, governments or governmental departments or agencies or their employees or agents; however, a Patient file(s) is/are always required and the system is patient-centric.

Any User may create a patient-centric electronic file(s) or data Storage by inputting said data types into the system or Application or Modules, which shall include but not be limited to fields storing or presenting the following information types: Patient, Medication, Behavioral, Biometric, Epidemiological, Environmental, Medical, Pharmacovigilance, Social, Social Media, or Virtual Data. In other embodiments, data types described herein may be put into the system via any Transmission method from any Source in various embodiments.

Patient Medications, and consumables (e.g., foods, beverages, etc.) are examined or Analyzed by the system to determine conflicts or negative or compounding or causal interactions (e.g., adverse drug reactions (ADRs)) between said items, or those Medications contra-indicated by age or other conditions, and upon discovering them, the system notifies the Patient, Patient contacts, and the Patient's treatment team or Health Care Providers or Users via one or more Transmission Methods of the discovery or results via alerts, push notifications to their email addresses, SMS text addresses, telephone numbers, mailing addresses, fax numbers, or direct-messaging addresses or other selected destinations or mediums including Output. The system may also send similar alerts or push notifications to Pharmaceutical Manufacturers, Insurance Carriers, regulatory authorities, governments or government agencies or public health agencies or groups.

Patients interact with one or more keyboards or User Interfaces (UI) to answer questions from predicative medication adherence surveys (e.g., including but not limited to Hogan Drug Attitude Inventory (DAI), Morisky Medication Adherence Scale (MMAS), Medication Adherence Rating System (MARS), etc.) or other tools using Internet-enabled Devices, Smart Devices or Wearable Computers, where after quantifiable scores, words, codes, numbers or metrics predicting future medication adherence, AEs or ADRs are recorded in Storage for Analysis, including but not limited to for the purposes of increasing or improving Patient or User awareness or mindfulness, or improving future medication adherence, clinical outcomes, cost efficiencies, cost effectiveness, population or public health or research. Such an embodiment may be repeated at frequent or infrequent, regular or irregular intervals and such results stored, processed, compiled, analyzed, and reported toward the same goals. Such an embodiment may also record such all types of data described herein in Storage for Analysis.

Users interact with one or more User Interfaces (UI) to select a customized or preferred schedule, grouping or cluster (e.g., around meal times, etc.), methods or mediums (e.g., email, SMS Text, direct messaging, telephone call, television subtitle or alarm, etc.), contacts (e.g., Patient, Patient family or contacts, Health Care Providers, or Users) and escalation sequence for Medication alerts or reminders or push notifications from the system or modules or applications.

Similarly, Users interact with one or more UI to select a customized or preferred schedule, method or medium, to receive confirmation notifications either in a meal-time grouping or at the individual medication level, that said Medications have been taken as directed, the answers to which are recorded in Storage for Analysis. When Patients are non-adherent to Medication, the system generates surveys or push notifications using a template wherein placeholders are replaced by actual values to elicit the reason(s) for non-adherence, which are recorded in Storage for Analysis, or acted upon via alerts to patients' health care team.

Patients or Users interact with one or more User Interfaces (UI) to select or input adverse events (AE) related to their Medication or dietary supplements in proximal time as to when the AE occurs or after the AE occurs, after which the system generates automated alerts or push notifications to family members, Medical providers, Users or public health or regulatory entities. The AE is also recorded in Storage for Analysis.

Users interact with one or more User Interfaces (UI) to answer questions (which may expressly include, but is not limited to, questions in the forms of narratives, numbers, symbols, avatars or emoticons) or select data fields or reasons for non-adherence or select or enter Behavioral, Biometric, Environmental, Epidemiological, Medical, Pharmacovigilance, Social, Social media or Virtual Data related to adherence or non-adherence to their medication regimens on Internet-enabled Devices, Smart Devices, or Wearable Computers where after quantifiable scores, responses or fields are Stored for Analysis or transmitted as Output.

Patients interact with one or more User Interfaces (UI) to set or modify digital rights management (DRM) for data (which expressly includes messages to or from them) related to them in Storage including, but not limited to: (a) who can receive, see or access their data (e.g., which Health Care Providers, Pharmaceutical Manufacturers, Insurance Carriers, or others); (b) how many times data can be viewed; (c) how long data can be viewed (e.g., sunrise and sunsets on the data—open to view, close to view, etc.); (d) setting conditions on who can view the data, how or when; (e) determining if their data can be forwarded and, if so, how many times or how long; (f) determining if their data can be printed and, if so, how many times, by whom or when; or, (g) rescinding any of the above rights.

In one embodiment, the invention and its systems and methods are comprised of three classes of Modules: (1) data modules; (2) processing or functional modules; and, (3) log modules (e.g., holding the data of what processing or functions occurred). Modules may include, but are not limited to, (a) Patient information; (b) Patient enrollment; (c) Notifications; (d) Safety or Pharmacovigilance; (e) Digital Rights Management; (f) Reporting or Output; (g) Data Input from Sources; and, (h) Learning or Analysis Module. The Modular examples listed above herein may have more than one type of module associated with it. For example, a module for learning or Analysis may have a module of each type—one for Storage of analyzed data, one for Analyzing data, and one for logging results of Analysis on said data. The scope of the invention for patenting here is inclusive of these module types for pharmacotherapy and medication management systems, the individual modules named, their collective, or any combination of them.

In another embodiment of the invention, the systems and methods are defined by the TRANSMISSION METHODS to one or more Modules receiving Behavioral, Biometric, Environmental, Epidemiological, Pharmacovigilance, Medical, Social, Social Media or Virtual Data inputs, for Storage, Analysis or Output. Such data includes, but is not limited to, identifying risks, conflicts or opportunities with Medication, predicting Medication adherence or non-adherence, improving or assisting Medication adherence, understanding why, when, or how Patients adhere, or fail to adhere, to their Medications, identifying or prioritizing or recording in Storage information pertaining ADRs or AEs for Analysis or to transmit in Output.

In another embodiment of the invention, the systems and methods are defined by the SOURCE from which one or more Modules receiving Behavioral, Biometric, Environmental, Epidemiological, Pharmacovigilance, Medical, Social, Social Media or Virtual Data inputs, for Storage, Analysis or Output. Such data includes, but is not limited to, identifying risks, conflicts or opportunities with Medication, predicting Medication adherence or non-adherence, improving or assisting Medication adherence, understanding why, when, or how Patients adhere, or fail to adhere, to their Medications, identifying or prioritizing or recording in Storage information pertaining ADRs or AEs for Analysis or to transmit in Output.

In another embodiment of the invention, the systems and methods are defined by the TYPE OF OUTPUT of Analysis from one or more Modules receiving Behavioral, Biometric, Environmental, Epidemiological, Pharmacovigilance, Medical, Social, Social Media or Virtual Data inputs, for Storage, Analysis or Output. Such data includes, but is not limited to, identifying risks, conflicts or opportunities with Medication, predicting Medication adherence or non-adherence, improving or assisting Medication adherence, understanding why, when, or how Patients adhere, or fail to adhere, to their Medications, identifying or prioritizing or recording in Storage information pertaining ADRs or AEs for Analysis or to transmit in Output.

The User Device operated by the Patient or other Users is a computer device, such as an Internet-enabled Device, Smart Device, or Wearable Computer. The User Device is used by Health Care Professionals to review a Patient's Medications, Biometric, Behavioral, Environmental, Epidemiological, Medical, Pharmacovigilance, Social, Social Media, or Virtual data expressly including adherence, survey results, ADRs or AEs and provide feedback, including but not limited to, coordinating care, answering and following-up with questions, or giving advice. The User Device executes an Application made up of one or Modules.

The invention, systems, methods, Applications, or Modules described herein are typically intended for use in post-market release (Phase IV) of Medications; however, in another embodiment, could be used during drug trials by Pharmaceutical Manufacturers or for Medications not regulated by governments to record in Storage for Analysis any of the data types described herein from the Sources or via the Transmission Methods or to produce Output.

In another embodiment, the invention, systems, methods, Applications, or Modules described herein could be used during military campaigns or operations, underwater, above the Earth or space explorations or activities, or remote monitoring or surveillance, to record in Storage for Analysis any of the data types described herein from the Sources or via the Transmission Methods or to produce Output.

In another embodiment, the invention, systems, methods, Applications, or Modules described herein could be used in hospitals, clinics, hospices, home health care, nursing homes, or ambulatory care centers to record in Storage for Analysis any of the data types described herein from the Sources or via the Transmission Methods or to produce Output.

In another embodiment, the invention, systems, methods, Applications, or Modules described herein could be used by manufacturers, owners, lessors, or operators of Medical Devices to record in Storage for Analysis any of the data types described herein from the Sources or via the Transmission Methods or to produce Output.

In another embodiment, the invention, systems, methods, Applications, or Modules described herein could be used by Pharmaceutical Manufacturers to record in Storage for Analysis any of the data types described herein from the Sources or via the Transmission Methods or to produce Output. Moreover, Pharmaceutical Manufacturers could use them to evaluate the efficacy, cost efficiency, cost effectiveness, ADRs, AEs related to their products. Moreover, Pharmaceutical Manufacturers could use them to identify new therapeutic opportunities and off-label uses of their products, alone and in combination with other Medications regardless of the manufacturer.

In another embodiment, the invention, systems, methods, Applications, or Modules described herein could be used by Health Care Providers to improve their pharmacotherapy management, medication adherence, care coordination, regulatory reporting, or clinical outcomes or conduct Analysis for research, treatment method or Medication efficacy or risks, ADRs, AEs, research or knowledge discovery related to the data fields.

In another embodiment, the invention, systems, methods, Applications, or Modules described herein could be used by technology or other companies to serve as part of or to supplement, or feed a larger computer system, artificial intelligence, deep learning, or semantic meaning analysis systems (e.g., including but not limited to IBM's Watson, Wolfram Alpha, DeepDive, Google's DeepMind, Microsoft's Cortana, MyA, MindMeld, HP Autonomy, Palantir, etc., or their successors) to facilitate improved pharmacotherapy management or efficacy, improved patient safety or treatment efficacy or options, or knowledge management, discovery, research or learning. Similarly, in another embodiment, the inventions described herein could be used by industrial or military organizations deploying exoskeletons that contain sensors to monitor and react to wearer or “pilots” Medical Data or Biomedical Data or physiological data.

In another embodiment, the invention, systems, methods, Applications, or Modules described herein could be used by Insurance Carriers or Payers to improve clinical outcomes, reduce costs, ADRs, or AEs, improve medication adherence, prevent hospitalizations or longer or more expensive treatment course or less efficacious treatment, identify risks or opportunities, reduce malpractice instances or claims or other types of torts, or conduct Analysis for product research or development, new or more efficacious treatment method, protocols, or regimens, or discovery knowledge, including but not limited to, about adherence, non-adherence, ADRs, or AEs.

In another embodiment, the invention, systems, methods, Applications, or Modules, data or Analysis described herein could be used by life science or pharmaceutical companies or research entities to for better targeting diagnostic-aided medicine, personalized medicine, drug targeting, product development, product improvement, delivery methods for Medications or therapeutics, biomarker-tagged products, engineered immunity, vaccines, or stratified therapy approaches.

In another embodiment, the invention, systems, methods, Applications, or Modules described herein could be used for post-marketing surveillance by Pharmaceutical Manufacturers, Health Care Providers, regulated parties (e.g. mandatory reporters, etc.) or government-related regulators or public health organizations to record in Storage for Analysis or Output ADRs or AEs to regulatory authorities or reporting systems or successors, (e.g., including but not limited to the US FDA Adverse Event Reporting System (FAERS), US Vaccine Adverse Event Reporting System (VAERS), EU Eudravigilance, European Medicines Agency, World Health Organization's (WHO) Program for International Drug Monitoring at Uppsala Monitoring Centre (UMC), China's Department of Drug Safety and Inspections (SFDA or BFDA), India's Central Drugs Standard Control Organization (CDSCO), Japan's Pharmaceuticals & Medical Device Agency (PMDA), or Ministry of Health, Labor & Welfare (MHLW), South Korea's Decentralized Pharmacovigilance System or Regional Pharmacovigilance Centers (RPVC), all national pharmacovigilance or public health surveillance agencies, etc.) or to be part of the chain of ADR or AE data flows. An extension or alternative to this embodiment allows governments or public health agencies or other interested and lawful parties to use the Application as a form of biological radar because it tracks the health status of patients by virtue of the medication regimens (and associated data including risks and efficacy) as it relates to other data from many Sources (e.g., physiological data, biometric data, etc.), that allows the Application to maximally temporally alert (e.g., as soon as known) infectious disease patterns or other public health emergencies.

In another embodiment, the invention, systems, methods, Applications, or Modules described herein, Health Care Providers could use it as a medication management system or mechanisms, processes, applications, or systems to improve their Star Rating by the US Center for Medicare & Medicaid Services (CMS) or other reimbursement or Payer programs.

In another embodiment, the invention, systems, methods, Applications, or Modules described herein, Health Care Providers could use it as a source for Electronic Health Records (EHR) or Electronic Medical Record (EMR) systems (e.g. including but not limited to eClinicalWorks', McKesson's, Cerner's, AllScript's, AthenaHealth's, GE Healthcare's, Epic, Care360, PracticeFusion, Optuminsights, Next Generation Health Care's, ADP's AdvancedMD, Soapwre, eMD's, Advanced Data System Corporation's, Vitera's, Meditab, Nuesoft, Greeway, AmazingCharts, OpenEMR, etc.) or their successors, or as a destination whereby these or other EHR or EMR systems or successors feed the invention to facilitate improved pharmacotherapy management or efficacy, improved patient safety or treatment efficacy, regulatory or other types of reporting, or knowledge discovery, research or learning.

In another embodiment, the invention, systems, methods, Applications, or Modules described herein, Health Care Providers, expressly including pharmacies, could use it as a source for electronic prescribing system (e.g., including but not limited to Surescripts, MediSecure, eRX, or successors etc.), transaction hub, computerized physician or provider order entry system (CPOE)(e.g., including but not limited to Cerner's CPOE or Millennium PharmNet, Clinicorp's, Eclipsys', MediTech's, Horizon's, McKesson's, PatientKeeper, CureMD, E-Medapps, Intivia, MedSym's, Netsmart Technology's, or succesors, etc.), computerized patient record systems (CPRS), patient or pharmacy management software (e.g., including but not limited to WinPharm, Liberty Software, PioneerRx, NRx, HBS Pharmacy Software, Lagnioppe, MediWork's, Abacus Pharmacy Plus Software, or successors, etc.), continuity of care system, computerized prescription systems, enterprise pharmacy systems (EPS) (e.g., including but not limited to PDX, etc.) or pharmacotherapy management or pharmacy information systems (e.g. including but not limited to Connexus, CPSI, HMS from Healthcare Management Systems, Inc., Siemen's Pharmacy, or successors, Netsmart's RXConnect, AllScript's Sunrise, etc.) or their successors, or as a destination whereby these pharmacy systems or successors feed the invention to facilitate improved pharmacotherapy management or efficacy, improved patient safety or treatment efficacy, regulatory or other types of reporting, or knowledge discovery, research or learning.

In another embodiment, the invention, systems, methods, Applications, or Modules described herein, Health Care Providers could use it as a source for diagnostic or clinical decision-support systems (e.g., including but not limited to Archimedes' IndiGO, Autonomy Health, DiagnosisOne—which became Alere Analytics which became Persivia, DXplain, Elsevier CDS, Isabel HCS, PKC, Micromedex, UpToDate, Provation Order Sets, BRCAPro, etc.) or their successors, or as a destination whereby these or other diagnostic or clinical decision-support systems or successors feed the invention to facilitate improved pharmacotherapy management or efficacy, improved patient safety or treatment efficacy, regulatory or other types of reporting, or knowledge discovery, research or learning.

In another embodiment of these systems and methods is a living, big data laboratory associated with Medications for data mining and knowledge discovery. Specifically, the collective data combined with the systems and methods articulated herein could be embodied in a personal data mining to identify relevant patient health information related to the correlations, efficacy, and consequences of Medication regimens that otherwise would likely remain undiscovered. Users and Sources supply Medication activity data that can be analyzed in conjunction with data associated with a plurality of other data, Medications, Analysis. Aggregated patient medication activity data can be mined on an individual basis or in conjunction with cohorts of other patients to identify correlations and/or causal probabilistic relationships (e.g. applying Bayesian methodologies, Bayesian networks, frequentist models, randomization, etc.) amongst the data and cohorts of patients and their medication activity behaviors, and health attributes. Applications or services can interact with such data and present it to users in a myriad of manners, for instance as notifications of opportunities or other Output.

In another embodiment, the invention, systems, methods, Applications, or Modules described herein, includes a pricing model system and method to charge fees associated with Digital Rights Management (DRM) activities (e.g. per encryption, decryption, print capability, forward capability, time-restricted viewing, quantity-restricted viewing—once, twice, etc.; and/or rescinding access) in healthcare management and pharmacotherapy per single file and/or message or use. Similarly, in a second embodiment, the invention allows Insurers, Payers, other Users to price the use or licensing of the Platform based on a dynamic pricing scheme wherein the licensing fee is computed based upon the higher or lower value a platform provides corresponding to more or less serious ailments or disease or the criticality and expense of individual medications in the regimen. For example, a regiment that included mission-critical and expensive oncology medications is license higher than a regimen made up of less life-critical or less expensive maintenance medications. In a third embodiment, and proprietary pricing or business or sales model, Payers are charged a value-add licensing fee that reflects a percentage of the savings they realize from its use. For example, the Platform is deployed by mandate by Payers to increase medication adherence, identify and notify members of patients' health care treatment teams of important information or events, prevent adverse reactions or events, or quickly response to adverse events or missed critical dosages thereby preventing tens of millions to billions of dollars of consequential health care costs that would require Payers to pay, and the ability to price the licensing of this and related or future technology on a value-add basis (e.g., 10% of the money annually saved the Payers).

The features and advantages described in the specification are not all inclusive and, in particular, many additional features and advantages will be apparent to one of ordinary skill in the art, in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF DRAWINGS

FIG. 1: Is a high-level diagram illustrating an embodiment of the logical architecture of a platform for managing medication therapy regimens, maximizing patient safety, coordinating care, improving clinical outcomes, regulatory reporting, and research and Analysis, according to one type of embodiment.

FIG. 2: Is a high-level diagram illustrating the relationship between the platform, components or modules, and the network(s) that provide communication infrastructure, according to one type of embodiment.

FIG. 3: Is a high-level diagram illustrating the technologies used for each functional layer of one embodiment of a platform for engaging patients, pharmacovigilance or coordinating care regarding medications, including but not limited to views, controllers or data stores or models, according to one type of embodiment.

FIG. 4: Is a high-level database schema for one embodiment of a platform for engaging patients, pharmacovigilance or coordinate care regarding medications, according to one type of embodiment.

FIG. 5: Is an algorithm flowchart for data and system decisions illustrating an embodiment of a platform for systems and methods for engaging patients, pharmacovigilance or coordinating care regarding medications, according to one type of embodiment.

FIG. 6: Is a block diagram illustrating an example interaction and relationships between Sources, Application, Transmission Methods, or Outputs for engaging patients, pharmacovigilance or coordinating care regarding medications, according to one type of embodiment.

FIG. 7: Is a block diagram illustrating an example interaction and relationships between Sources, Applications, and Output as part of a Health Care Provider system, according to one type of embodiment.

FIG. 8: Is a block diagram illustrating an example interaction and relationships between Sources, Applications, and Output, according to one type of embodiment, as part of a research and development system employing data warehouses, Storage, and Analysis.

FIG. 9: A block diagram illustrating an example interaction and relationships between Sources, Applications, and Output, according to one type of embodiment, for public health surveillance and regulatory reporting.

FIG. 10: A block diagram illustrating an example interaction and relationships between Sources, Applications, and Output, according to one type of embodiment, as part of a larger computer system, artificial intelligence (AI), or semantic meaning analysis system (SMAS) (e.g., IBM's Watson, Google Now, Wolfram Alpha, etc.).

FIG. 11: A user interface (UI) example illustrating how Patients may access, enroll, set, or display Patient Data or personal information, according to one type of embodiment.

FIG. 12: A user interface (UI) example illustrating how Patients may access, enroll, set, or display contact methods, including but not limited to SMS Text, Smart device, or direct messaging (e.g., WhatsApp, Facebook Messenger, etc.), including but not limited to addresses or information, according to one type of embodiment.

FIG. 13: A user interface (UI) example illustrating how Patients may access, enroll, set, or display emergency contact information, according to one type of embodiment.

FIG. 14: A user interface (UI) example illustrating how Patients, Providers, Industry or other users may log-into the SaaS platform, according to one type of embodiment.

FIG. 15: A user-interface (UI) example illustrating a system or method for a Patient dashboard, according to one type of embodiment.

FIG. 16: A user-interface (UI) example illustrating how Patient may set, access, prioritize, customize, group, or display reminder notifications for Medications, according to one type of embodiment.

FIG. 17: A user-interface (UI) example illustrating how Patient medication regimens may be set, edited or displayed, according to one type of embodiment.

FIG. 18: A user-interface (UI) example illustrating the system or method for patients to report adverse events (AEs) or reactions, according to one type of embodiment.

FIG. 19: A user-interface (UI) example illustrating a system or method for Patients to set, edit, or customize their health care treatment team of Providers, or others on their care treatment team (e.g., home care providers, next of kin, etc.), according to one type of embodiment.

FIG. 20: A user-interface (UI) example illustrating a system or method of displaying or presenting or administering surveys or assessments that quantify and predict a patient's risk of medication non-adherence on the screen of any Internet-enabled device, and enabling a User to quickly and easily answer questions, and communicate them back to the Application, according to one type of embodiment.

FIG. 21: A user-interface (UI) example illustrating a system or method for a Provider (e.g., physician, pharmacist, etc.) dashboard, according to one type of embodiment

FIG. 22: A user-interface (UI) example illustrating a system or method of displaying, setting or controlling medication regimen across a cohort of Provider or Industry patients, according to one embodiment.

FIG. 23: A user-interface (UI) example illustrating a system or method of displaying, editing, or controlling assessments or surveys predicting medication non-adherence of a cohort of patients, according to one embodiment.

FIG. 24: A user-interface (UI) example illustrating a system or method of displaying or controlling information pertaining to a cohort of Patients for Providers or Industry, or loading information (e.g., an API UI), according to one embodiment.

FIG. 25: A process-flow or flowchart algorithm illustrating a system or method for pharmacovigilance—identifying and reporting drug-drug interactions or drug-food interactions—according to one embodiment.

FIG. 26: A process-flow or flowchart algorithm illustrating a system or method for quantifying or evaluating or surveying, calculating, or alerting actual medication regimen adherence or non-adherence, according to one embodiment.

FIG. 27: A process-flow or flowchart algorithm illustrating a system or method for quantifying or evaluating or surveying, calculating, or alerting predicted medication regimen non-adherence, according to one embodiment.

FIG. 28: A process-flow or flowchart algorithm illustrating a system or method for identifying, describing, alerting, or displaying adverse reactions or events, according to one embodiment.

FIG. 29: A process-flow or flowchart algorithm illustrating a system or method for a vertically integrated Big Data as a Service (BDaaS) stack including, but not limited to, the relationships between technologies including for data services, business intelligence, or Analysis or visualizations, according to one embodiment.

FIG. 30: A process-flow or flowchart algorithm illustrating a system or method for Analysis, data mining, knowledge discovery, related to the data types described herein and pharmacotherapy, according to one embodiment.

FIG. 31: A process-flow or flowchart algorithm illustrating a system or method for Analysis, data mining, knowledge discovery, related to the data types described herein and pharmacotherapy, including data integrated or aggregated from disparate sources, according to one embodiment.

FIG. 32: A process-flow algorithm and description illustrating a rules-based inference engine for complex event processing (CEP) related to any and all patient behaviors directly or indirectly related to the patient's medication regimen (e.g., medication-taking behaviors or medication-related events), or any and all other physiological or environmental responses related to the patient's medication regimen or health.

### DETAILED DESCRIPTION OF DIAGRAMS & DRAWINGS

The invention at a detailed functional or operational level consists of systems, methods, and computer programs to perform the following possible functions: (a) monitor Patient adherence to a complete Medication regimen; (b) quantify or predict Patient non-adherence to Medications; (c) customize alerts or reminders or push notifications for Medications based on groups, timing or priority at the Medication or dosage level of detail; (d) customize alerts or reminders or push notifications for Patients to consume Medications based on editable rules, for example, an escalating method of contact to determine both the methods (e.g., email, SMS Text, direct messaging, phone call, etc.), the number of reminders, their sequence, and the parties to contact; (e) coordinate care amongst Patients, patient families, Health Care Providers, Pharmaceutical Manufacturers, Insurance Carriers, or Payers related to Medication regimens, Medication interactions, Medication adverse events, efficacy or risks by Patient, or predicted clinical outcomes; (f) Store, or Analyze Biometric, Behavioral, Epidemiological, Environmental, Medical, Social, Social Media, or Virtual data related to Medication regimens and/or patient health and/or Medication efficacy or risks by Patient and/or predicted health outcomes; (g) connect, link or intercommunicate between Wearable Computers, Internet-enabled devices, sensors, or other Sources to the Application and other systems; (h) integrate calendars and time data with Medication regimens, predictions, activity and/or inactivity and relate it to Patient health or lifestyle, life satisfaction, Medication efficacy or risks, or quality or predicted clinical health outcomes; (i) generate and render interactive events or elements combining Medication activity/inactivity or Medication efficacy or risks by Patient with all data from all Sources; (j) operate a software application and/or platform to engage patients, maximize Medication safety and efficacy, coordinate care as associated with multiple Providers, manage medication regimens and/or predicted health outcomes; (k) set or manage the digital security rights (DRM) of patient Medication activity/inactivity, Medication efficacy or risks by Patient, Behavioral, Biometric, Epidemiological, Environmental, Medical, Social, Social Media, or Virtual data as related to Medication regimens, Patient health or predicted or real clinical outcomes; (l) User Interfaces for communicating information, interacting, and navigating as related to Medication regimens; (m) to allow Users to program and connect to Wearable Computers, Internet-enabled Devices, Internet-of-Things Devices, or Smart Devices customizable cascades, arrangements or sequences, related to Medication regimens; (n) to learn or discover knowledge or information about patient users' environments, behaviors, risks or needs related to Medication regimens, or predict or execute their preferences automatically (e.g., connecting inventions related to Medication management to a smart watch to a smart thermostat to predict the time at which a medication that gives a patient chills is to be taken and making their environment warmer in preparation at the given time; or, connecting inventions related to medication management here to a smart refrigerator to ensure a patient taking anti-depressants deletes grapefruit-products from the refrigerator inventory and/or ordering system—therein, enhancing the experience, optimizing machines, quantifying the self, extending safety and security, minimizing interaction risks, and outcomes of medication adherence and healthcare treatment); (o) allow Users to quantify or self-measure their Medication activities (e.g., adherence, non-adherence, Adverse Events, Adverse Reactions or interactions, or Medication risks or efficacy under different scenarios or circumstances, etc.) and juxtapose their Medication activities to others; and, (p) collect, record, Store, Analyze, report, or communicate Medication and Medication-related information to, between, and from Wearable Computers, Internet-enabled Devices, Smart Devices, or Internet-of-Things Devices, including but not limited to, between Patients, Providers, Payers, Pharmaceutical Manufacturers, and creators or purveyors of health information technology or research; (q) escrow data keeping a copy of critical Application data allowing clients to protect and insure all data that resides within one embodiment of the Application is protected against data loss.

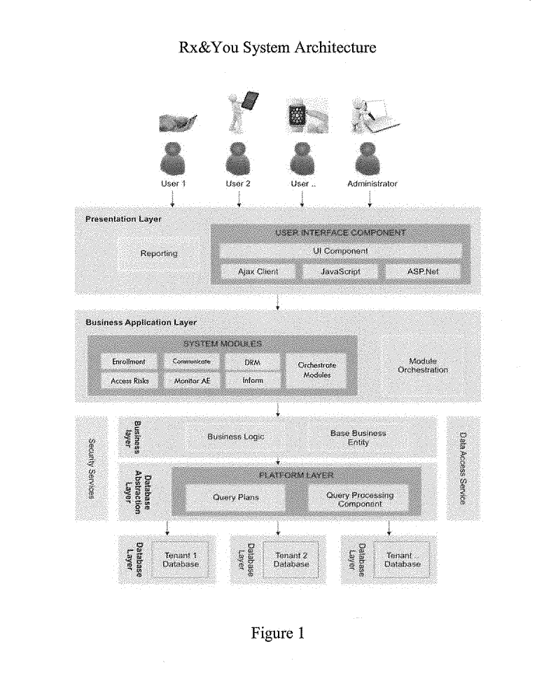

FIG. 1: A system (or Platform) architecture diagram of a Complex Event Processing (CEP) artificial intelligence system delivered as Software as a Service (SaaS) via Cloud Computing which, according to one type of embodiment, consists of a modified form of Open System Interconnections (OSI) architecture consisting of a presentation layer, business application layer (encompassing security and data access as “cousins”), a platform layer, or data base layer. In this embodiment, the Application is a multi-tenanted architecture typically installed on multiple machines for horizontal scaling, hosted in and deployed from a central location. This exemplary embodiment has a single configuration and an application programming interface (API) as an open integration protocol for feeds from Patients and all the Data Sources defined herein.

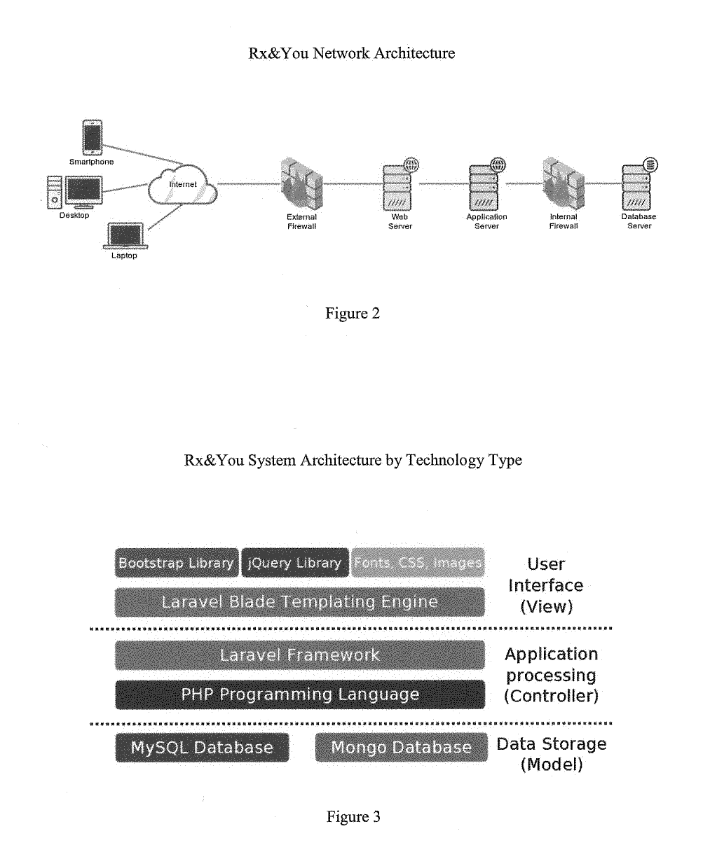

FIG. 2: A network architecture diagram, according to one type of embodiment, illustrating a network communication consisting of a framework of physical components and their functional organization and configuration, which consists at a sub-component level of an Internet Protocol Suite, and includes interconnecting networks and nodes. The platform environment includes a server(s), firewalls(s), a Patient User Device, and other Users' Devices (e.g., Health Care Providers, Pharmaceutical Manufacturers, Insurance Carriers, Payers, etc.). Only one server system, one Patient User Device, and one other User's Device (a Healthcare Provider in this example) are illustrated; however, in practice, there may be multiple instances of each of these entities. For example, there may be millions of Patient User or Internet-enabled Devices in connection with tens of thousands or more of Healthcare Provider or Payer User Devices and numerous server system(s).

FIG. 3: A system architecture by product or technology type, according to one type of embodiment, which consists of a layered framework of technology products organized by function and showing their relationship to each other. In this embodiment, the Platform functions as a cohesive distributed systems architecture making it a totally integrated system of disparate technologies, development platforms, products and services.

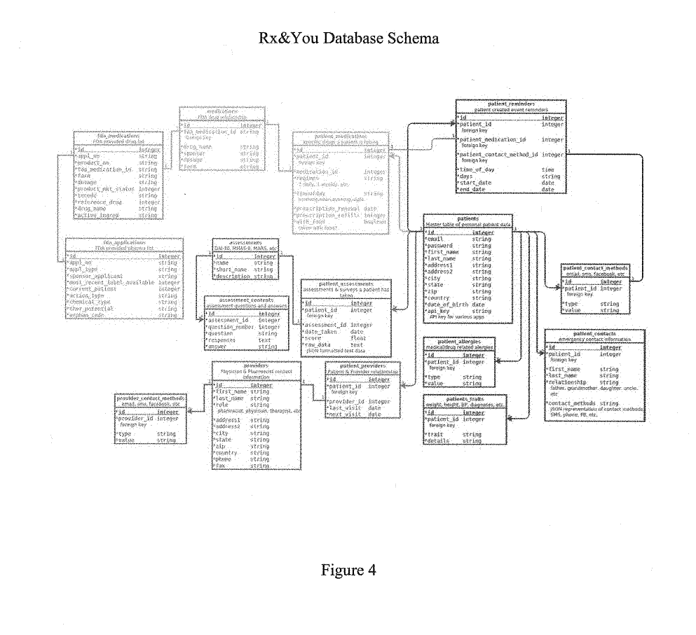

FIG. 4: A database schema that, according to one type of embodiment, illustrates a logic and organization to group objects, here data types, fields, and tables, along with the relationships between fields and tables (e.g., a relational database). It is the databases structure, defined in a formalized graphical language (e.g., a schema as depicted herein) supported by a database management system (MySQL according to this embodiment and illustration but could also be variations thereof, NoSQL, etc.) that is proprietary and acts as a blueprint for the organization of data. In other embodiments, data pools or lakes, data marts or warehouses, may exist in addition to or in lieu of group objects in the logic and organization illustrated here.

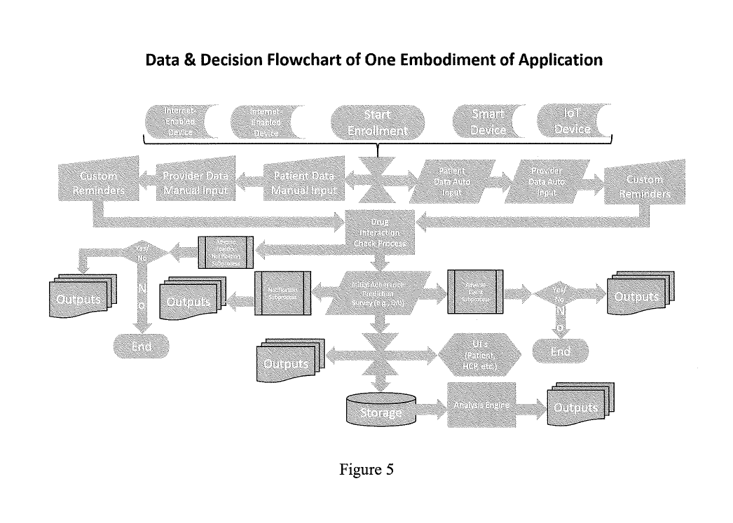

FIG. 5: An algorithm flowchart that, according to one type of embodiment, displays how data flows through the Platform and how decisions are made to control events. Symbols are used to designate Platform start and end points (ovals), processes (plain rectangles), sub-processes (rectangles with vertical lines), decision points (diamonds), data or files (parallelograms), output documents (rectangles with curved bottom), output displays (oblong hexagons), manual (rectangle with angled top) versus automatic inputs (left facing concave-ended “bullet” shapes), collation points to organize data (an “hourglass”), and data flow directions (arrows). It represents a collection of proprietary flow chart algorithms or workflows, and graphically symbolizes major inputs, processes, and outputs.

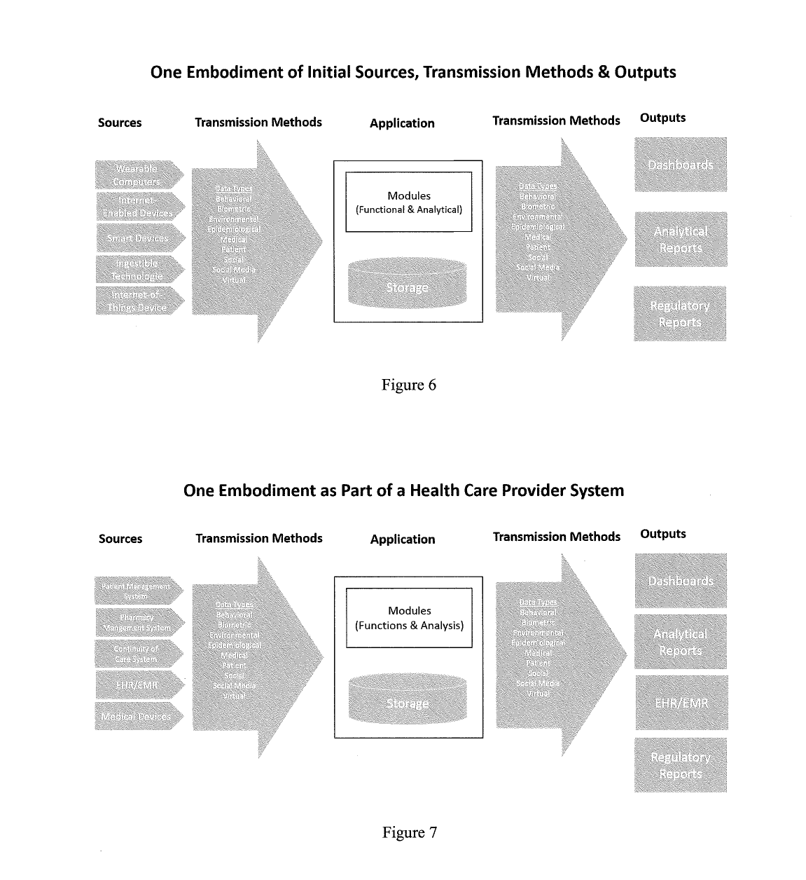

FIG. 6: A block diagram illustrating data flows and the relationships between possible Sources, Transmission Methods of data types, the Platform or Application, and Outputs according to one embodiment. In this instance, data is manually inputted by patients or automatically loaded by one or more types of Internet-enabled computers or devices via our proprietary Application Programing Interface (API). The Platform adds value via the systems and methods described herein, then uses one or more Transmission Methods to present the results in one or more Output forms to Patients, Healthcare Professionals or Providers, Pharmaceutical Manufacturers, Payers, or other Users.

FIG. 7: A block diagram illustrating data flows and the relationships between possible Sources, Transmission Methods of data types, the Platform or Application, and Outputs according to one embodiment. In this instance, the Application acts as a destination, Source, or value add as part of a larger or wider Healthcare Provider system wherein Electronic Health or Medical Records (EHR/EMR) or Medical Devices may act as both a Source or Output, along with or in place of Patient Management Systems.

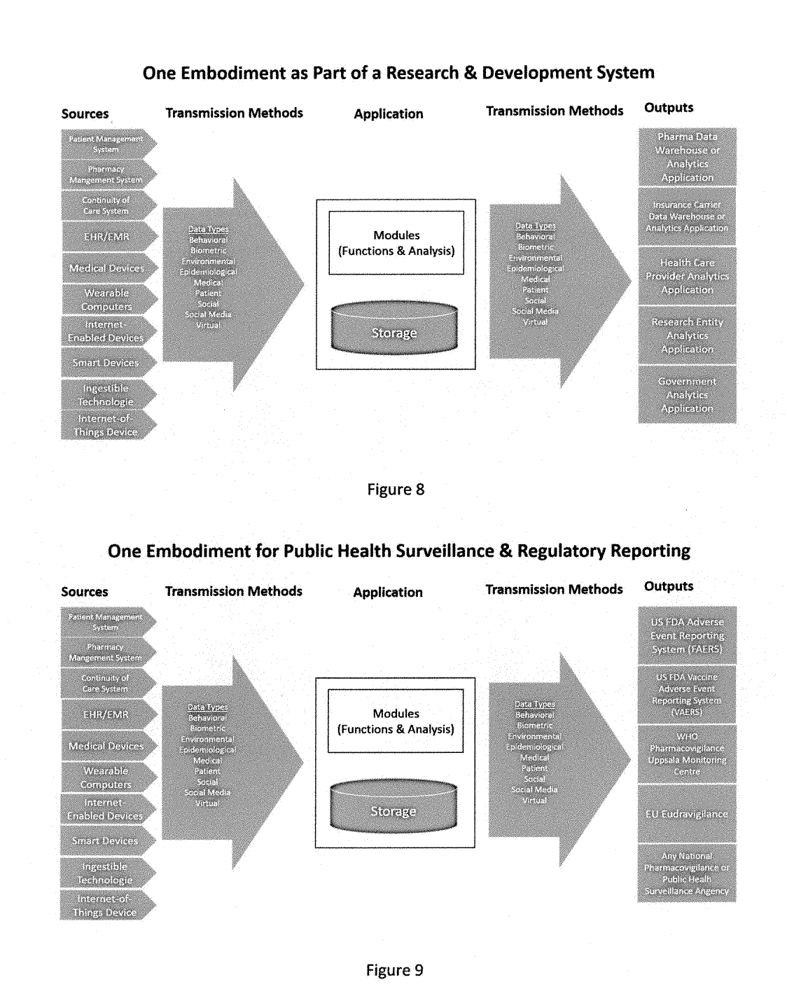

FIG. 8: A block diagram illustrating data flows and the relationships between possible Sources, Transmission Methods of data types, the Platform or Application, and Outputs according to one type of embodiment. In this instance, the Application acts as a destination, Source, or value-add processor or analyzer, as part of a larger or wider research and development system for consumers, Healthcare Providers, Pharmaceutical Manufacturers, Insurance Carriers, Payers, public health agencies or groups, laboratories, universities, or other research entities. In this embodiment, for example, the Outputs would feed a data warehouse, data mining, or analytics engine for product development, studies, deep learning, or knowledge discovery (Note, these capabilities also may exist in within an embodiment of the Application or Platform). Thereby, third-party Users could conduct this research, development, deep learning, data mining, knowledge discovery, or Analysis with value-added data, Analysis, and Output from the Application or Platform, or have the owners of the Platform or Application conduct that research, development, or Analysis on the Users' behalf in a fee-for-service pricing or business model.

FIG. 9: A block diagram illustrating data flows and the relationships between possible Sources, Transmission Methods of data types, the Platform or Application, and Outputs according to one type of embodiment. In this instance, the Application acts as a Source, or value add, for the purpose of reporting or transmitting data to public health agencies for surveillance or research entities or for the purposes of regulatory reporting to governmental or quasi-governmental agencies (e.g., reporting Adverse Events (AE) to the US FDA Adverse Event Reporting System (FAERS), or the US FDA Vaccine Adverse Event Reporting System (VAERS), or World Health Organization's (WHO) Uppsala Monitoring Centre, or other national or international public health monitoring or surveillance systems, studies, or applications).

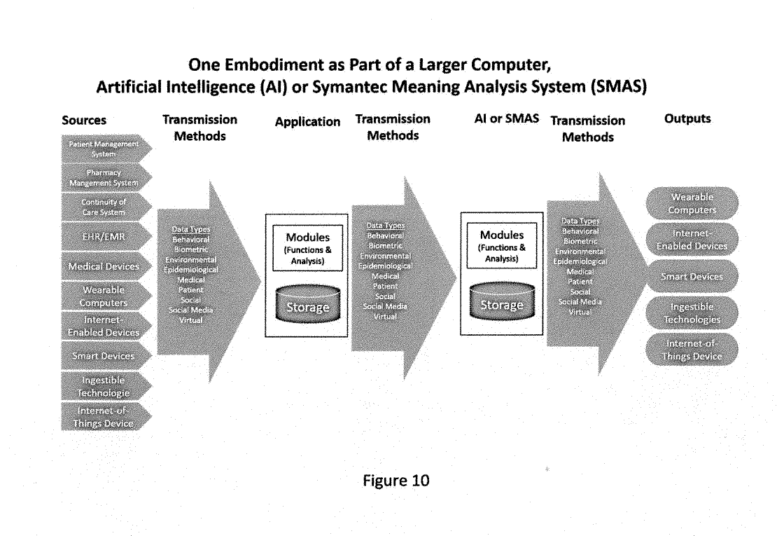

FIG. 10: A block diagram illustrating data flows and the relationships between possible Sources, Transmission Methods of data types, the Platform or Application, and Outputs according to one type of embodiment. In this instance, the Application acts as a Source, or value-add processer or analyzer, as part of a larger computer, Artificial Intelligence (e.g., IBM's Watson, Microsoft's Project Oxford, Google's DeepMind, Baidu Minwa, etc.), Deep Learning, semantic meaning networks or systems, machine learning (e.g., Microsoft's Azure, etc.), neural-networking, deep neural-networks, or cognitive-computing systems. In this embodiment, computational knowledge or answer engines are the recipient of Users' queries or computational requests via a text field or voice demand (e.g., asking Apple's Siri or Microsoft's Cortana a question by spoken words), where after the knowledge or answer engines compute answers or relevant visualizations from a knowledge base of curated, structured data and/or unstructured data, including that which comes from other databases, electronic sources, websites, or books. In this embodiment, the Application or Platform is acting as such a Source to provide aggregates or Analysis of any of the types of data it collects relative to Patient actions or reactions (e.g., patients from Iceland under the age of 35 often have an adverse reaction to Medication X, etc.). Another application of this embodiment is the Platform, or its value-add Analysis Outputs, serving as a Source for the analysis, preparation, or Output of “personal analytics” reports or profiles containing an analysis of the user's Medication outcomes or relationships relative to their social, on-line or other activities (e.g., Wolfram Alpha using the profile of a Facebook user containing analysis of the user's social relationships and activities relative to their Medication regimen, events, risks, or efficacy).

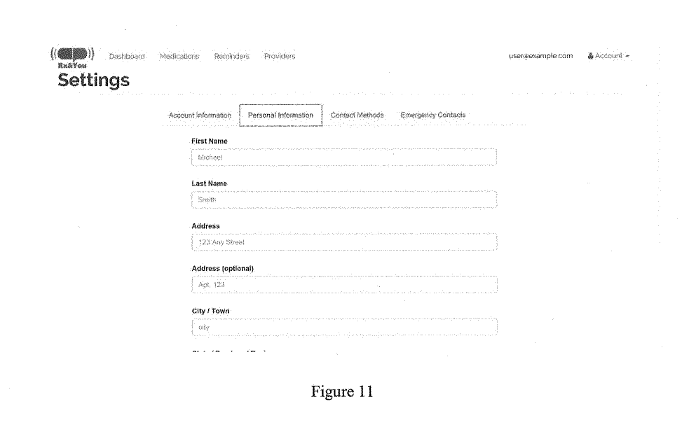

FIG. 11: A screen capture illustrating a User Interface, according to one embodiment, to allow Patients to enter or edit their personal or contact information.

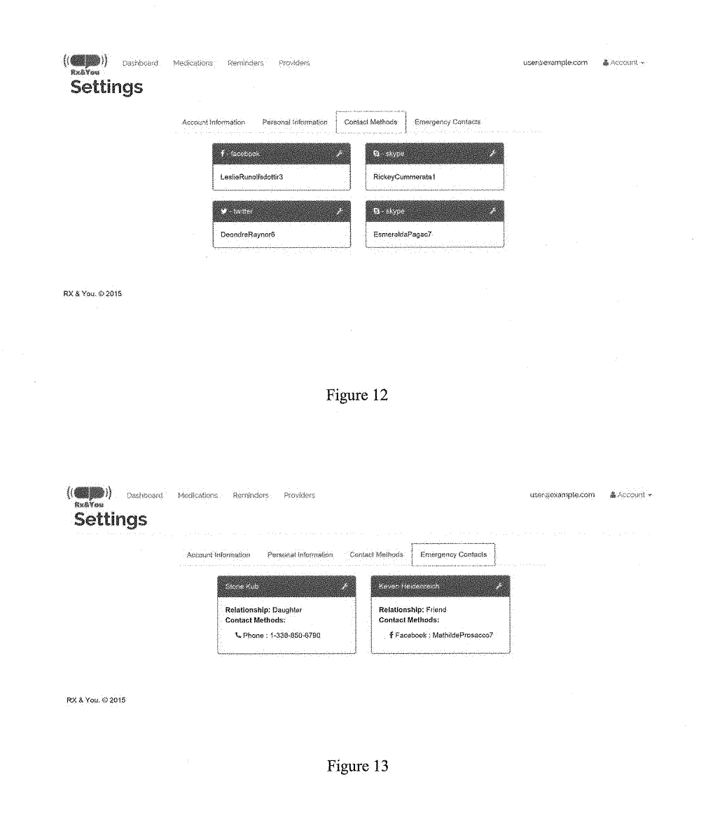

FIG. 12: A screen capture illustrating a User Interface, according to one type of embodiment, to allow Patients to enter or edit the family members, emergency contacts, caregivers, or healthcare providers names, contact information or methods for the purposes of receiving alerts, reminders, or push notifications, coordinating or communicating with their Healthcare Providers or treatment team.

FIG. 13: A screen capture illustrating a User Interface (UI), according to one embodiment, to allow Patients to enter or edit direct messaging (DM) addresses (e.g., WhatsApp, Facebook Messenger, WeChat, etc.) or other contact information, for the purposes of receiving alerts, reminders, or push notifications, coordinating or communicating with their Healthcare Providers or treatment team. Proprietary here is the system or method of grouping or clustering Medication taking and reminding events, in this embodiment, by meal times, after awaking, and before sleeping.

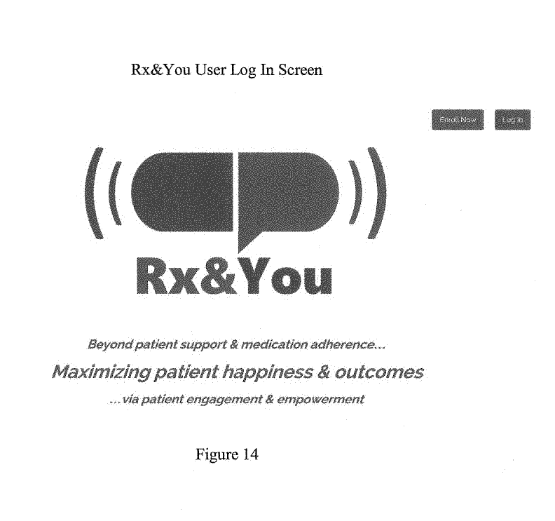

FIG. 14: A screen capture illustrating an example of a User Interface (UI), according to one type of embodiment, that allows Users to log-on to the Cloud-based SaaS (or BDaaS) AI Platform, including but not limited to the ability to identify users by type (e.g., Patient, Provider, Payer, Industry, etc.) thereby directing them to different data views, screen collections, presentations, and functions that are specific to their needs or interests (which may function similar to a “cookie” or “super cookie”). The same UI example may allow new Patients (or their Providers', or representatives) to enroll in the platform, whereby after clicking the “Enroll” button, the prospective User is taken through a series of registration screens, most if not all of which, are depicted as examples herein. As a practical matter, Users are expected to be enrolled in large bulks automatically via a proprietary Application Programing Interface (API); however, Users can be enrolled individually if and when desirable.

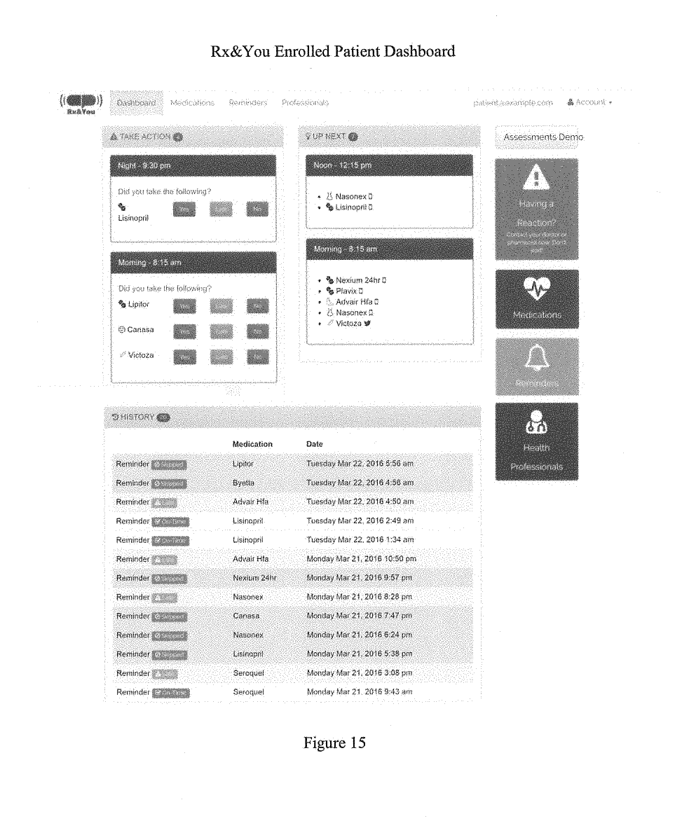

FIG. 15: A screen capture illustrating an example of a User Interface (UI), according to one type of embodiment, that allows Patients already enrolled in the program to have a dashboard “starting place” or “landing page” that may depict: (1) recent historical medical regimen activity (illustrated here on the left side); (2) near-term future medical regimen activity (illustrated here on the right side); and, buttons (right side) or drop-down menus or links (top of screen) for major functions (illustrated here with Adverse Event, Reminders, Medications, Providers). Depictions may include, as they do in this example, a proprietary cross-cultural color coding for positive events (e.g., green), cautionary events (e.g., yellow), or adverse or negative events (e.g., red). Depictions may also include symbols or icons for the type or reminder alert a Patient or User has selected (e.g., Twitter “bird,” mobile phone, Skype direct-message, etc.). A third type of depiction is symbols or icons for the medium of medication (e.g., tablets, capsules, nasal mists, eye drops, etc.) to help Users confirm and verify it is the correct Medication. The system also allows Patients to edit or modify events. In other embodiments, the dashboard will allow coordination of care that goes beyond Users of different types logging in to review medical concordance (e.g., working from one regimen list across Providers) to include communication modules that allow messaging between Patients and Providers and between Providers, including storage and retrieval of said messages), and file up-loading and sharing, the Digital Rights Management for which Patients may set pertaining to their own records depending on the record, recipient or Provider, time, access rights and privileges, etc.). Said file sharing expressly includes, but is not limited to, Medical Data, medical files, PDFs, records, scores, etc. to enhance the coordination of care and maximize drug safety and clinical outcomes across Providers, treatment teams, maladies, regimens, and time.

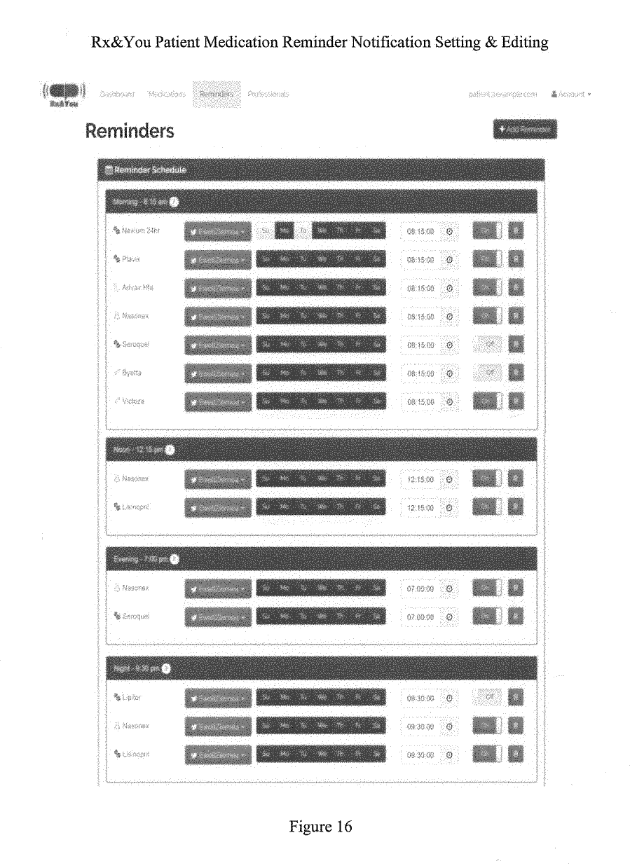

FIG. 16: A screen capture illustrating a User Interface (UI), according to one type of embodiment, for Patients to input or manage their Medication regimen reminders or alert notifications according to a customizable schedule, including but limited to the day of the week, the hour of the day, dosages, or quantities, refill date(s), methods and addresses for reminder alerts or push notifications, including but not limited to, the sequence of escalation of such notifications (e.g., email me 30 minutes before Medication due, SMS text me five minutes before, call my mobile telephone if I have not confirmed taking it via a UI within 10 minutes after due, etc.). Included herein is the proprietary ability to cluster or group reminders or Medication-taking behaviors, for example here, when awaking, meal times, or going to sleep at night.

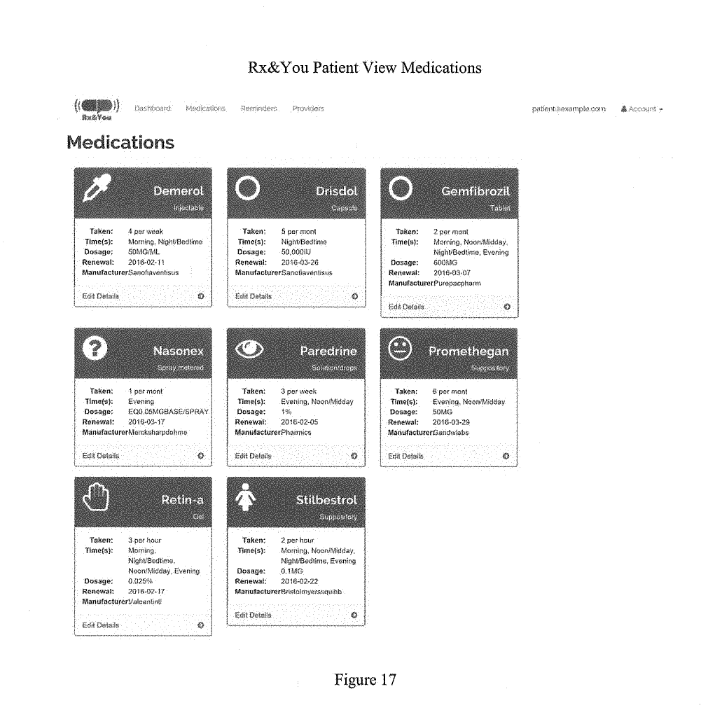

FIG. 17: A screen capture illustrating a User Interface, according to one embodiment, for Patients to input or manage their Medications, including but limited to the name of the Medication, the form in which it is taken or prescribed, the quantity, dosage, or refill date. All these feeds are editable by the Patient or User representing the Patient.

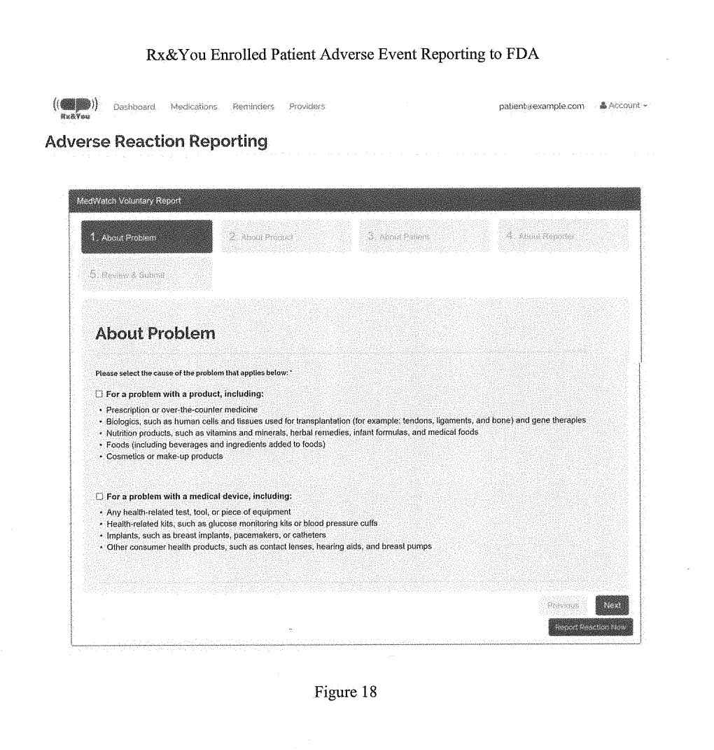

FIG. 18: A screen capture illustrating an example of a User Interface (UI), according to one type of embodiment, that allows Users (typically Patients, however, Users in all instances could also include caregivers, patient family members, or Providers) to report an adverse drug event (ADE) through one or more questions, through one or more screens. In this particular example, said reporting is anonymized dependent upon the recipient of subsequent alerts or notifications (e.g., the FDA may be alerted but would not necessarily know the Patient's identifying information; similarly, Pharmaceutical Manufacturers may be notified without knowing the identity of the patient, only metadata about the patient). Embedded behind the series of screens with which this example begins is functionality that alerts a Patient's treatment team (e.g., physician(s), pharmacists, care givers, emergency contacts, etc.) as to the fact that the Adverse Event is occurring and its nature.

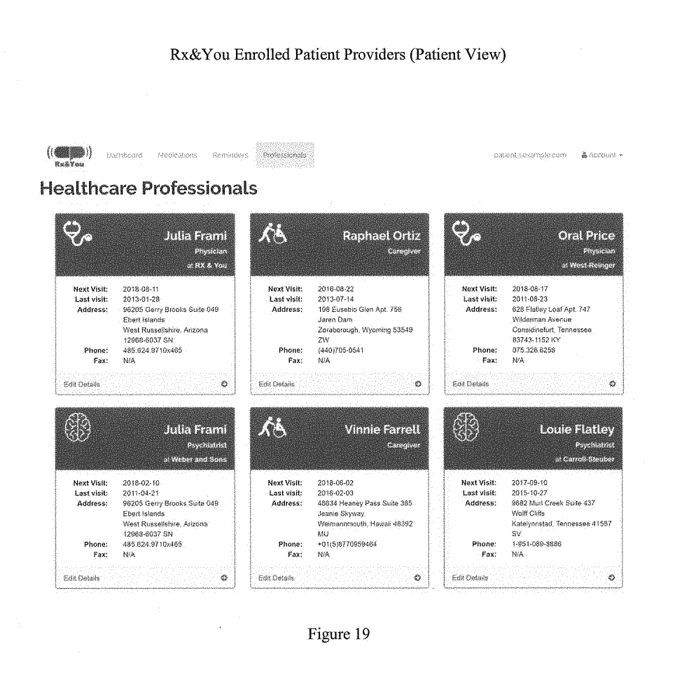

FIG. 19: A screen capture illustrating an example of a User Interface (UI), according to one type of embodiment, that allows Users (typically Patients, however, Users in all instances could also include caregivers, patient family members, or Providers) to identify their Providers or treatment team through one or more questions, through one or more screens. In this example, Users are able to enter, store, or edit information pertaining to identity, location, and contact information preferred for each provider (e.g., e-mail addresses, SMS Text numbers, direct messaging addresses, telephone numbers, etc.). This information can be used by the platform to coordinate care and maximize patient safety including by messages, alerts, shared files or Medical Data, and medication regimen or medical treatment concordance.

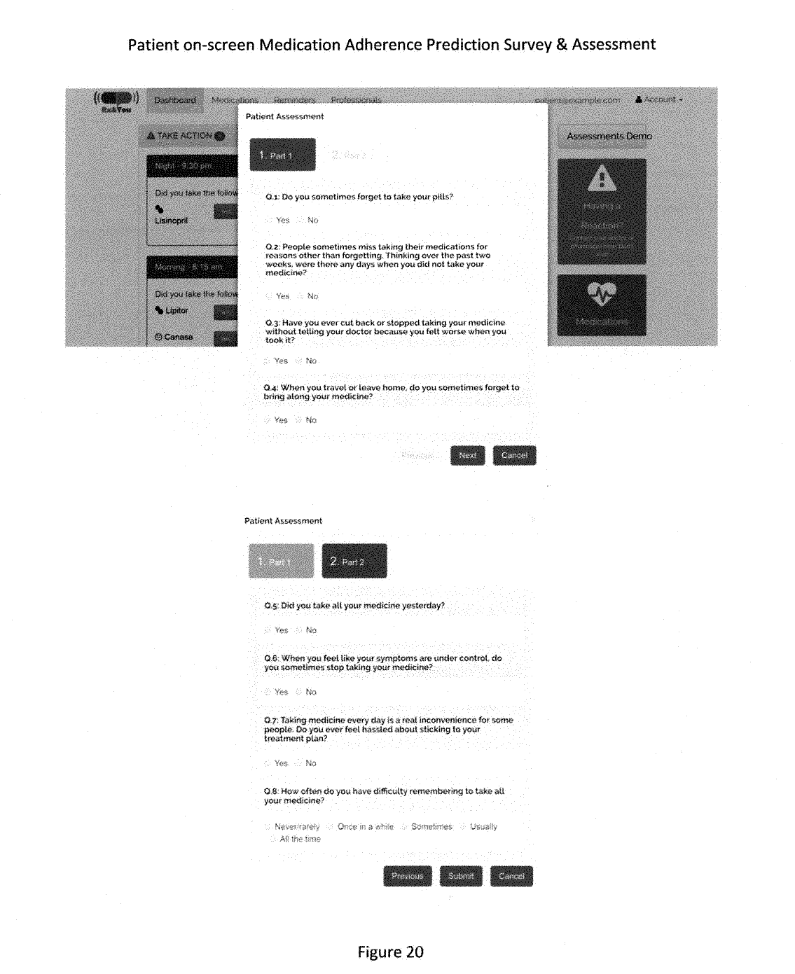

FIG. 20: A screen capture illustrating an example of a User Interface (UI) that, according to embodiment, allows surveys or assessments, used to quantify or predict medication non-adherence, to be presented to Users (typically Patients) on a screen of any Internet-enabled device whether seen when a User log-ins to the Application on a computer (e.g., desktop, laptop, tablet, etc.) or sent as a push notification to an Internet-enabled device (e.g., Smartphone, Smartwatch, etc.), which also allows the User to intercommunicate with the Application by answering concise questions via radio buttons or drop-down menus or buttons, and sending the feedback (an event) to the Application to Store, prioritize, math, Analyze, act upon or Report. Moreover, the system includes a proprietary method by which, when a Patient User selects the red-colored button for not having taken a medication, the Patient User is presented with a short list of reasons why they did not take their medication which after selecting, the Platform also sends this data (another event) to Store, prioritize, match, Analyze, act upon or Report. In such a manner, the Platform records a date-time stamped series of events about the Patient's medication regimen-taking behavior that includes the Medication, dosage, adherence, quality of adherence (late or timely) and/or reasons for non-adherence, which the Platform can Store, track, prioritize, match, Analyze, act upon, or Report over time to any authorized User.

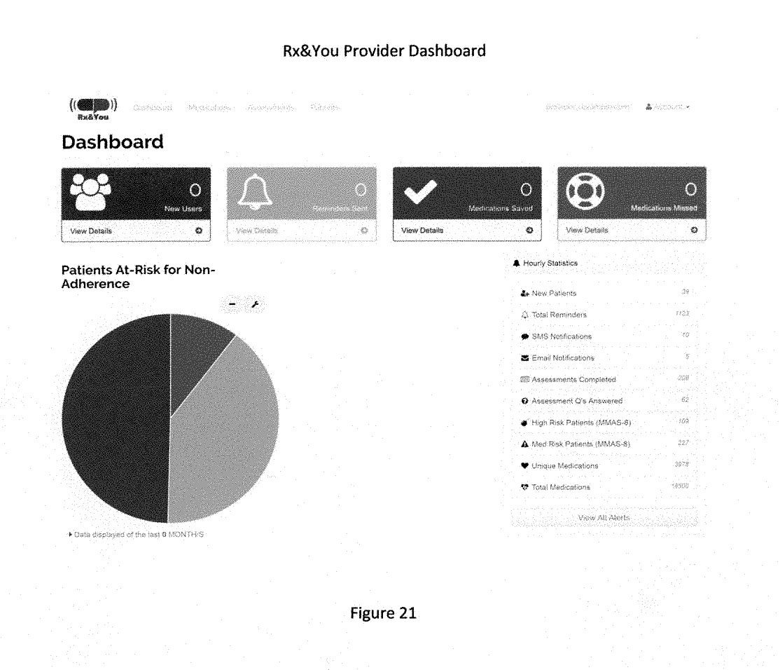

FIG. 21: A screen capture illustrating an example of a User Interface (UI), according to one embodiment, that allows Users (typically Providers such as physicians or pharmacists) a dashboard used as a “starting place” or “landing page” after Providers log-in to see the number of Patients enrolled in their cohort (attached to their services), the total number Medications those Patients use, and which Medications their Patient cohort has been prescribed. This User Interface, according to one embodiment, can also be used by industrial Users (e.g., including but not limited to Pharmaceutical Manufacturers, Insurance Carriers, Hospital Corporations, Payers, etc.) to display aggregate data or Analysis as Output in which they have a vested interest (e.g., quantity of patients adhering by geography, age, gender, Medications; or, types or frequency of Adverse Events (AEs) or Adverse Drug Reactions (ADRs) by patient types).

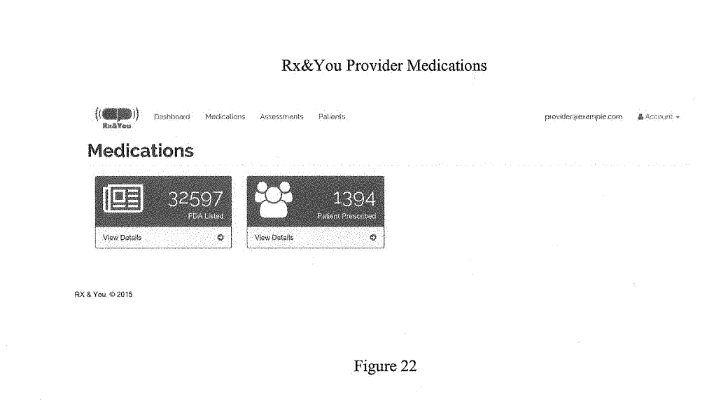

FIG. 22: A screen capture illustrating an example of a User Interface (UI), according to one type of embodiment that allows Users (typically Providers such as physicians or pharmacists) a dashboard to see information pertaining to Patient Medications. In this example, the total number of Medications in that User's “view” of the Platform or Application (from the FDA here), and the total quantity of Medications that Provider Use has prescribed, issued, or is tracking across their cohort of Patients. Such a cohort may represent, in one embodiment, the Patients of an individual Provider (e.g., physician, pharmacist, psychiatrist, etc.), or Provider group (e.g., a medical practice or retail pharmacy), or corporate Provider (e.g., .a hospital or pharmacy chain). In this embodiment, by clicking “Details,” a Provider User may see breakdowns of types of Medications in the Platform, or various distributions of Medications in its cohort (e.g., types of Medications written or filled or being used, by gender, geography, time, location, efficacy, adverse events, interactions, etc.).

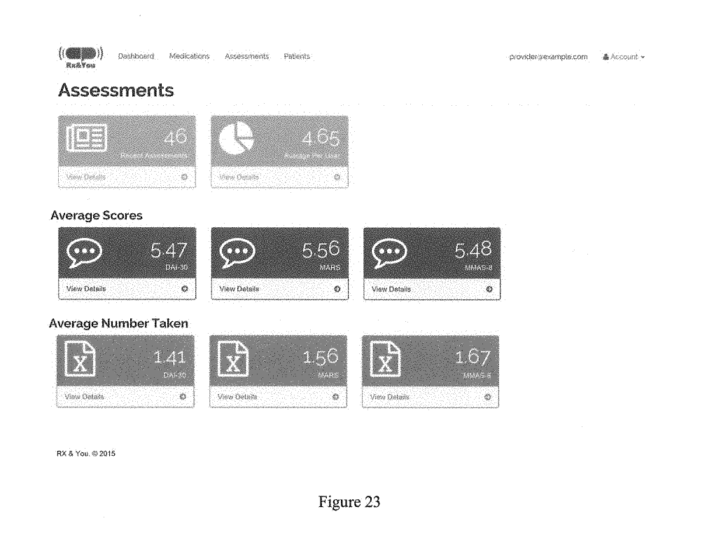

FIG. 23: A screen capture illustrating an example of a User Interface (UI), according to one type of embodiment, that allows Users (typically Providers such as physicians or pharmacists) a dashboard to see information pertaining to medication adherence surveys, assessments, scores, and activities. In this example, Users may see the total number, frequency and type of assessments or surveys their cohorts of Patients have taken, their average scores, and profiles or tabulations communicating which Patients, or types of Patients, are adhering or not adhering to their Medication regimens, and predictions, probabilities, or risks of future adherence or non-adherence. Provider Users may also click “View Details” in this embodiment to see the types of Patients, Medications, times, or locations where medication non-adherence is occurring, or to track any of these data over time. In this embodiment, using the rule-based inference engine depicted in Drawing or Diagram 32, alerts or notifications may also be set regarding Patient's survey or assessment scores. For example, this information may be used by a Provider to identify which Patients require additional time, attention, or treatment to follow prescribed protocols, or the reasons why they are or are not following their regimens. Another embodiment may also show, for example, the reason(s) why a Patient is non-adherence or at risk of being non-adherent, their Adverse Events (AEs) or drug interactions or risks. Another embodiment may also allow Providers or Industry to evaluate the efficacy of the Medication regimen, or predict future efficacy based on past performance, genotypes, or data from other Sources. In this embodiment, cross-cultural color-coding continues to be used wherein green indicates a positive or adherent event, yellow indicates a cautionary event (e.g., quasi or late adherence), and red indicates a negative or unwanted event.

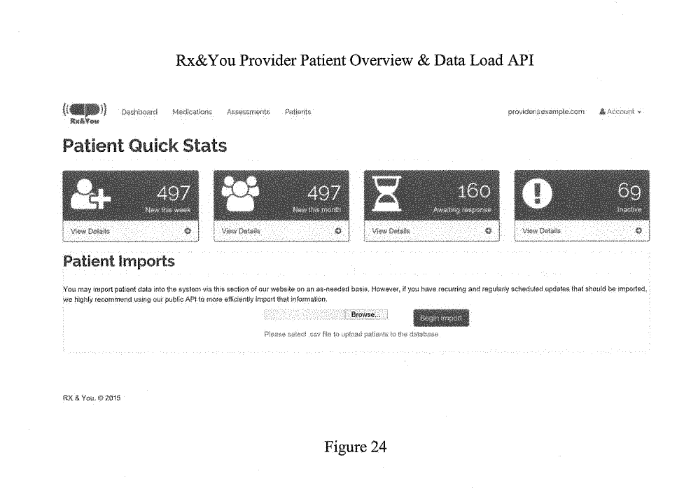

FIG. 24: A screen capture illustrating an example of a User Interface (UI), according to one embodiment, that allows Users (typically Providers such as physicians or pharmacists) a dashboard containing data related to Patient numbers, activities, and regimens. For example, in this illustration, Providers may see the total number of Patients they have enrolled that have an affiliation with them (their cohorts), and their enrollment status in the program (e.g., total enrolled, new enrollees last time period, number of Patients waiting to be enrolled, number inactive or declined or exited the enrollment process, etc.). In this embodiment, by clicking on “View Details,” a Provider User may see varying and customizable distributions of the data in greater granularity related to times, locations, gender, Patient ages, and Medication types, and other demographics or cohorts that the Provider User may define. This embodiment also contains functions showing the quantity (and type if desirable) of Patients in queue to use the Platform, and who, if any, Patient Users have opted out of using the Platform. This embodiment also contains a button to trigger a proprietary Application Programming Interface (API) that allows Providers (or Payers or other Industrial Users) to load cohorts (groups) of Patients to the Platform.

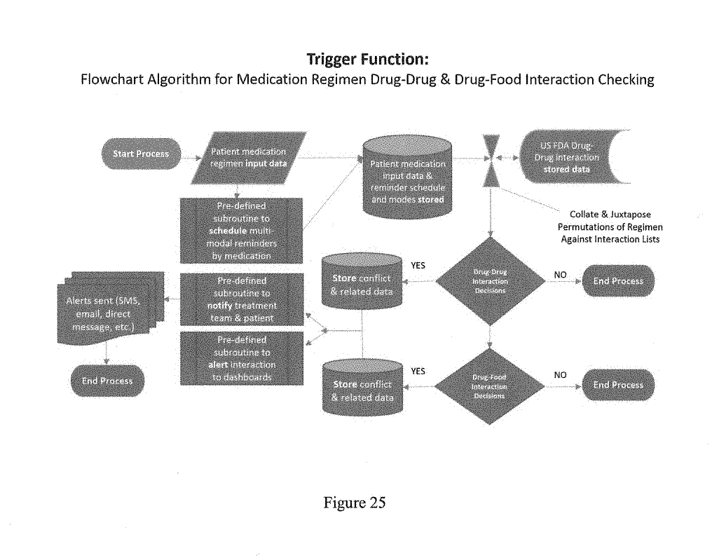

FIG. 25: A process flow or flow chart algorithm illustrating a system or method, according to one embodiment, for processing, analyzing, deciding, quantifying, storing, and alerting or notifying drug-drug, drug-food, or drug-other type of interactions. These interactions could include, but not be limited to, FDA warning about drugs or foods or items that should never be taken together, said items that could have a negative or positive compounding effect, or interactions that are not yet identified until Analysis by the platform. In this example, each Analysis and decision related to interactions is a separate and related process that involves permutations that exceed human cognitive abilities across whole Medical regimens. Another embodiment is interaction checking related to genotypes or family medical histories, which may include Analysis that predicts the probability that a certain Patient or Patient profile will have an efficacious, beneficial, or harmful experience with a particular Medication or regimen relative to their genetic, proteinomics, biome, family history, or any of the other types of data defined herein from any Sources. Another embodiment of the same processes and methods is for prescription drug fraud prevention by identifying Providers and/or Patients with duplicate prescriptions, prescriptions over the same time period, or redundant functions.

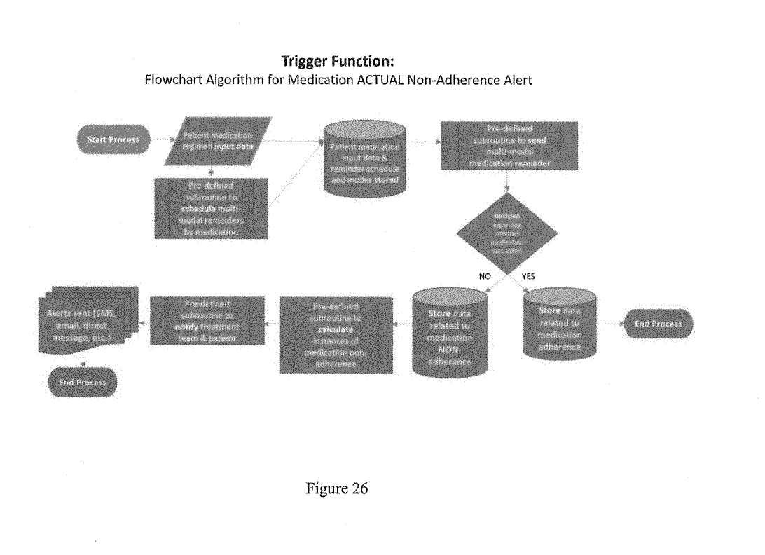

FIG. 26: A process flow or flow chart algorithm illustrating a system or method, according to one type of embodiment, for identifying, quantifying, recording, and alerting as to Patients' actual (e.g., instead of predicted) adherence to Medication regimens. For example, a Patient taking a 10-drug regimen may have 10-20 events per day to schedule, remind, confirm, and tabulate, which this process accomplishes by the multiple steps and methods indicated. It expressly includes the ability to customize the trigger-points of alerts (e.g., some medications may require immediate alerts for missed does, such as heart medications, others may be notified or alerted only when three or more doses are missed in a given period of time). It expressly includes the ability to customize the recipient and method of communication of alerts (e.g., caregivers may want notification of important Medications missed, physicians or pharmacists for others, with different levels of communication based on priority—e.g., SMS Text for missed high-priority Medications and emailed before office visit for lower-priority Medications or alerts).

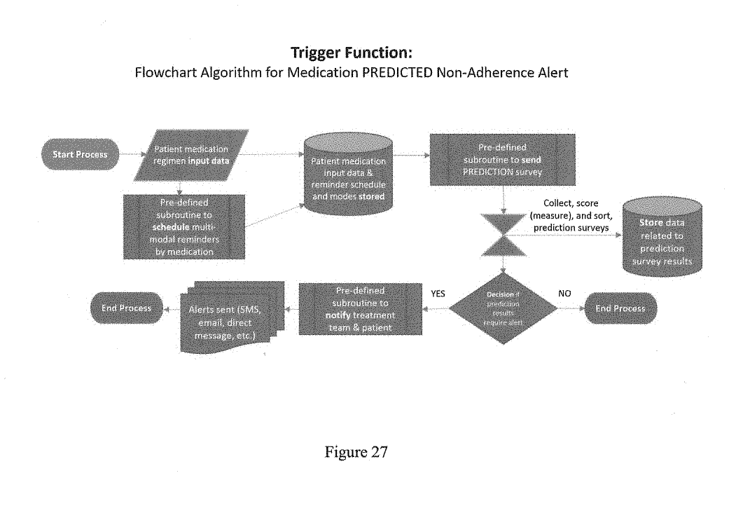

FIG. 27: A process flow or flow chart algorithm illustrating a system or method, according to one embodiment, for identifying, quantifying, recording, and alerting as to Patients' predicted (e.g., instead of actual) adherence to Medication regimens. For example, in this embodiment, three predictive surveys are presumed (Hogan DAI-30, Morisky, and MARS), which are administered by these systems and processes to Patients as they enroll (or log-in for the first time), and on a recurring basis the trigger for which is customizable as to time based, event based, etc.). The system or method is inclusive of any and all predictive surveys, of which there are at least 43 at time of filing, and is not limited to the three examples used in this embodiment. The system or method includes the ability to display, present, administer, intercommunicate, store or track predicative surveys or assessments over time for each Medication or a combination of Medications (e.g., regimen), to inquire, store, Analyze, and notify or alert as to reasons for higher assessment scores. It expressly includes the ability to customize the assessment(s) given depending on the contents of the Medication regimen (e.g., MARS is typically given if the regimen includes mental health drugs). It expressly includes the ability to customize the recipient and method of communication of alerts (e.g., caregivers may want notification of higher predicative assessments for important Medications missed, physicians or pharmacists for others, with different levels of communication based on priority—e.g., SMS Text for missed high-priority risks and emailed before office visit for lower-priority Medications or alerts). As a point of clarification, the System or Method excludes copyrightable content (the actual verbiage) of surveys or assessments written by other parties but comprehensively includes systems and methods to display, present, intercommunicate, quantify, administer, store, organize, process, analyze or report survey or assessment Data from all Sources related to pharmacotherapy management, Medication risks or efficacy.

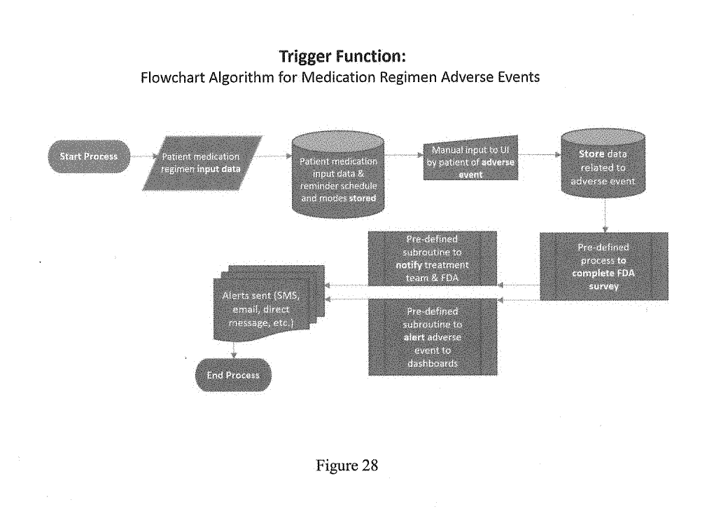

FIG. 28: A process flow or flow chart algorithm illustrating a system or method, according to one embodiment, for identifying, quantifying, recording, and alerting as to Patients' adverse events (AEs) to Medication regimens. Patients, or other Users, are stepped through this process via multiple screens (as illustrated earlier herein) of key questions Providers, Industry, or the FDA wishes to know about an adverse event, then prepares and sends custom notifications, the content of which is largely based upon the recipient (e.g., Industry and FDA is anonymized; however, Providers need to know when a Patient is having an adverse event with as much specificity as possible). It expressly includes the ability to stratify adverse events based on priority or importance and treat them differently (e.g., notifying a caregiver by phone call versus email a record to a pharmaceutical manufacture for more evaluation). It expressly includes the ability to customize the content of each and every alert to include, for example in other embodiments, genetic, proteinomics, biome, family history or other Data types from other Sources herein as part of the profile and notification or alert).

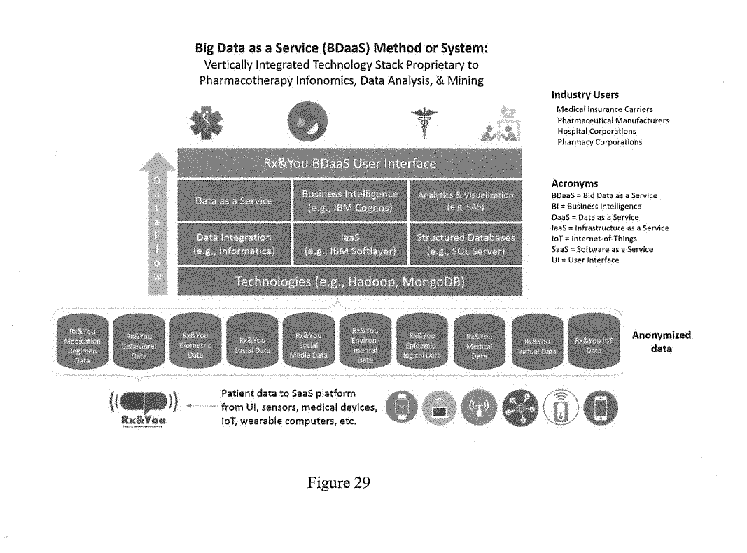

FIG. 29: A block diagram illustrating a system or method, according to one embodiment, for providing Big Data as a Service (BDaaS) to Users (typically but not always Insurance Carriers, Hospital Corporations, Pharmacy Corporations, or Pharmaceutical Manufacturers) in a vertically integrated way that allows all Data from the platform to be made available as a Data Service, for Business Intelligence, or Analysis or Visualization via proprietary User Interface(s) via technology stacks that combine proprietary and commercial technologies. In this embodiment, Data from the invention and all other Sources is expressly included. This embodiment may include data warehouses, data lakes, structured, or unstructured data stored, tabulated, organized, combined, aggregated, or Analyzed in such a way as to discover associations, mine data, and support infonomics (the discovery, provision, and sale of information).

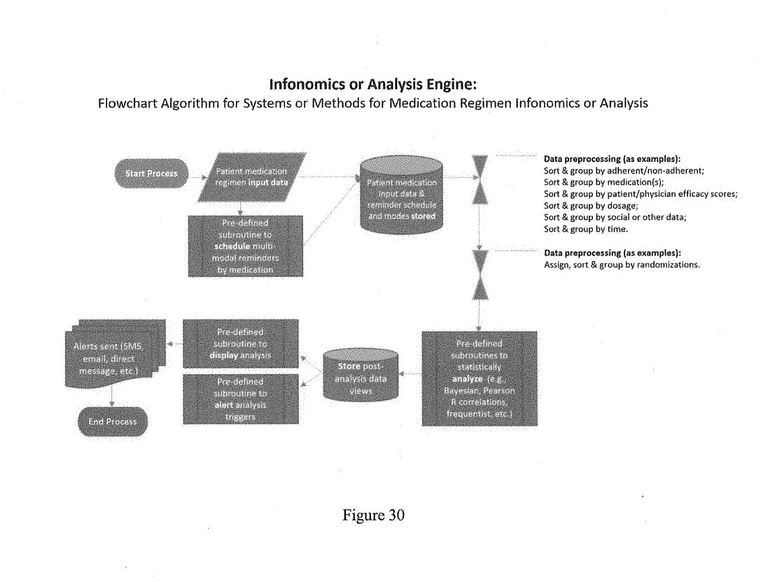

FIG. 30: A process flow or flow chart algorithm illustrating a system or method, according to one embodiment, for an Analysis engine to identify, tabulate, Analyze, store, extrapolate, notify or alert data patterns, unknown influencers, correlations and relationships between all Data types in the platform. This system and method allows for data integration that is already part of the platform or system and is, primarily, already structured or organized.

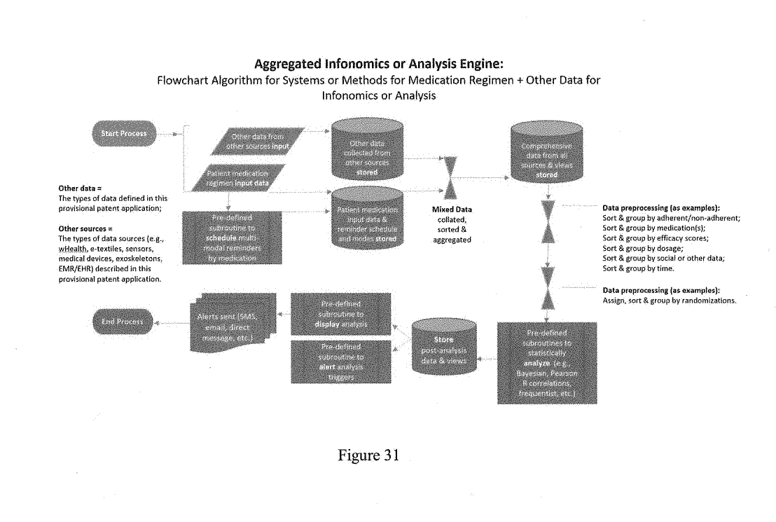

FIG. 31: A process flow or flow chart algorithm illustrating a system or method, according to one embodiment, for an Analysis engine to identify, tabulate, Analyze, store, extrapolate, notify or alert data patterns, unknown influencers, correlations and relationships between all Data types from all Sources. This system and method allows for aggregation and integration of disparate sources and types of data for Analysis.

FIG. 32: A process flow algorithm illustrating a system or method, according to one embodiment, for a rule-based inference engine to identify, prioritize, match, analyze and act for complex event processing (CEP) wherein every interaction a patient has with their medication across the regimen is treated as a behavioral event to be so treated by the inference engine. Moreover, said engine processes Data Types as events from all Sources for the same purposes. Actions may include but are not limited to telephoning, texting, direct messaging, or e-mailing appropriate member(s) of a patient's health care treatment team. It also includes a user interface that allows the creation of new rules and actions and the editing of existing priorities, Data Types, Sources, and Actions.

### Definitions Used Herein

“Analysis” or “Analyze” as used herein is defined to include, but not be limited to, causal or descriptive or exploratory or examination or evaluation or inferential or mechanistic or predicative analysis or study or relationship mapping of the Behavioral, Biometric, Environmental, Epidemiological, Medical, Pharmacovigilance, Social, Social Media, or Virtual data elements and/or their structure(s), also including but not limited to correlations, causal (e.g., Bayesian) relationships, calculating, counting, summing, measuring, mathematically or statistically manipulating or processing, comparing, juxtaposing, contrasting, tabulating, or extrapolating any combination of those data elements, data mining, data exploration, models, knowledge discovery, conversion, or consolidation of any combination of said data elements or objects;

“Application (or “App”)” as used herein is defined to include, but not be limited to, any self-contained program or piece of software designed to fulfill a particular purpose or purposes; at times, the terms “Platform” or “System” are used interchangeably with the term “Application;”

“Behavioral Data” as used herein is defined to include, but not be limited to, behavioral information related to a patient, or cohorts of patients, expressly including medication adherence/non-adherence, diet, physical activity or inactivity, fatigue, insomnia, pain, sleep patterns, emotional states (e.g., angry, depressed, manic, etc.), and any and all combinations and/or computations of these data points (e.g., averages, extrapolations, rates of change, etc.) or Analysis;

“Biometric (or E-health) Data” as used herein is defined to include, but not be limited to: heart rate, respiration rate, airflow breathing, electrocardiogram (ECG), oxygen in blood (SPO2) levels, CO2 levels, blood pressure (systolic and/or diastolic), galvanic skin response (GSR to quantify sweating), patient position (from an accelerometer, glucose levels, sleep patterns, body temperature, muscle electromyography (EMG), blood chemistry data, hydration, feedback from ultrasound pads, feedback from breast tissue sensors, speed of movement, body mass index (BMI), weight, feedback from sensors measuring UV radiation, fertility or menstrual cycles, caloric burn, pedometers, peak flow sensors, brain sensors (e.g., EEG), feedback from microarrays, ECG beat detection, ECG QRS detection, QT intervals, T waves, U waves, and any and all combinations and/or computations of these data points (e.g., averages, extrapolations, rates of change, etc.) or Analysis;

“Digital Rights Management (DRM)” as used herein is defined to include, but not be limited to, a systemic approach to protecting digital media to prevent unauthorized use or distribution and to restrict the ways recipients or accessors of digital files may use the data as set by the data owner(s) (e.g., typically in the instant invention it will be set by Patients; however, in certain circumstances, other data ownership types and DRM setters could apply).

“Electronic Health Record (EHR)” or “Electronic Medical Records (EMR)” as used herein is defined to include, but not be limited to, any electronic version of a patient's medical history, including those maintained over time, demographic, identity data, biometric data, progress notes, prescriptions, behaviors, reactions, medications, or problems whether proprietary to a Health Care Provider or owned or licensed by a for-profit or not-for-profit company;

“Environmental Data” as used herein is defined to include, but not be limited to, information to or from any device or sensor communicating, expressly including those communicating wirelessly and/or optically, measuring ambient temperature, air quality, barometric pressure, geographic location, altitude, weather, precipitation, wind, noise levels, drift detection, moon phases, tides and currents, weather patterns, and any and all combinations and/or computations of these data points (e.g., averages, extrapolations, rates of change, etc.) or Analysis;

“Epidemiological Data” or “Pharmacovigilance Data” as used herein is defined to include, but not be limited to, that related to patient safety and medications, expressly including drug-to-drug conflicts, drug-to-food conflicts, drug-to-beverage conflicts, dosage-to-weight conflicts, dosage-to-age conflicts, dosage-to-gender conflicts, dosage-to-genetic permutation conflicts, adverse drug reactions (ADR), delayed diagnosis and/or treatment, errors in diagnosis, failure to prevent, errors in drug usage, sepsis or septicemia, viremia, respiratory failure, heart failure, organ failure, cancer, complications, adverse events (AE), symptoms, side effects, and any and all information related to the initiation or abatement of such items and/or combinations and/or computations of these data points (e.g., averages, extrapolations, rates of change, etc.) or Analysis;

“Health Care Provider” (or “Provider”) as used herein is defined to include, but not be limited to, hospital corporations, medical practices, physician(s), psychiatrist(s), psychologist(s), pharmacist(s), pharmacies, nurses, nurse practitioners, physician's assistant, hospitals or employees or agents or contractors, surgery centers, clinics, urgent care facilities or practices, ambulatory care centers or employees or agents or contractors, nursing homes or employees or agents or contractors, hospice providers or employees, doctors of medicine, doctors of osteopathy, functional medicine doctors or providers, optometrists, chiropractors, podiatrists, midwives, counselors, therapists, dentist, orthodontist, health-care worker, health maintenance organization, or any other health related service provider whether corporate or individual;

“Ingestible Technologies” as used herein is defined to include, but not be limited to, smart pills, ingestible sensors, and edible or drinkable, medical devices;

“Internet-enabled Device” or “Smart Device” as used herein is defined to include, but not be limited to, personal computers, laptops, keyboard, mouse, graphical or video or motion-sensing or holographic interface, voice interpreter or translator, tablet computers, notebook computers, smartphones, heads-up displays, projections, or any electronic device with a User Interface (UI) and processor that can transmit information to or via the Internet, Intranet or Transmission Methods or their successors, whether not connected to or technically using the public Internet or successors (e.g., it includes electronic devices that exchange or transfer all data types defined herein by any and all Transmission Methods);

“Internet-of-Things (IoT) Device” as used herein is defined to include, but not be limited to, any and all devices that can communicate to or via the Internet, Intranets, or Transmission Methods or their successors, including but not limited to appliances, vehicles, monitors, thermostats, gauges, scales, lighting, meters, security systems, food packaging, beverage packaging, medication packaging, diagnostic devices, printers, scanners, personal care items (e.g., toothbrushes, etc.), gaming consoles, televisions, radios, cameras, video cameras, public infrastructure, hospitality infrastructure, medical infrastructure, retail infrastructure, roadways, transportation or mobility aids, casts, bodily sensors, sensor suits, satellite, submersible device, watercraft, armor, drones, aircraft, surveillance equipment, consumer devices, or any other inanimate object that is assigned an Internet Protocol (IP) addresses (e.g., including but not limited to IPv6, etc.) in any version or their successors;

“Insurance Carrier” or “Payer” or “Payor” as used herein is defined to include, but not be limited to, a company, group, municipality, government, or individual that offers, underwrites, or sells insurance policies, that pays for healthcare services or products (expressly including national health services, US Medicare, Medicaid, etc.), employers so engaged, community organizations so engaged, or captive insurance entities;

“Medical Data” as used herein is defined to include, but not be limited to, medication information related to a patient, or cohorts of patients, expressly including blood chemistry, data related to heart or brain or respiratory tract or circulatory or optical or renal or immune or gastrointestinal or endocrinological or dermatological or ocular or reproductive or nervous system, data related to health or family history, psychiatric and/or psychological health, substance abuse, developmental history, psychosocial or sociocultural history, occupational and/or military history, problems, reactions, Medications, progress notes, diagnostic results, images, scans, blood chemistry, electrolytes, hydration status, toxin or Medication exposure, microbiome, cellular sequencing or activity, genetic, proteinomics (including dynamics and folding), DNA, RNA, mRNA, tRNA, rRNA, scaRNA, snoRNA, single nucleotide polymorphisms (SNPs), major histocompatibility complex(es) (MHC), antibodies, vaccine status or exposure, data related to a person or persons' biome (bacterial flora), disease status or exposure, study results or participation, disease exposure, diagnostic test result(s), photographs, recordings, videos, sounds, and any and all combinations, interactions, and/or computations of these data points (e.g., averages, extrapolations, rates of change, correlations whether Bayesian or otherwise, etc.) or Analysis;

“Medical Device(s)” as used herein includes, but is not limited to, wirelessly or optically communicating implants and/or electronic sensors, glucose monitors, blood chemistry monitors, heart pacemakers, pumps, cardioverter defibrillators, breast implant sensors, brain sensors, central nervous system sensors, spine fusion hardware, intra-uterine devices, implants, shunts, graphs, traumatic fracture repair hardware, artificial knees or hips, coronary stents, tympanostomy (ear) tubes, psuedophakos (artificial eye lenses), hearing aids, electronic sensors, biosensors, flow sensors, image sensors, retinal implants, cochlear implants, glaucoma sensors, intracranial pressure sensors, motion sensors, mood sensors, magnetoencephalography (MEG) and magnetocardiography (MCG) systems using superconducting quantum interference devices or SQUIDs, encoders, gurneys, meters, gauges, tables, bodily suits with sensors, diagnostic computers or software, imaging machines, scanners, body analyzers, blood analyzers, MEMS sensors, pressure sensors, thermometers, blood pressure measuring systems, DNA sequencers, nanopore sequencers, microfluidic devices, polymerase chain reaction (PCR) devices, any device that is extracting information from or stores or analyzes data related to the patient's body, microfluidic capillary electrophoresis (CE) devices, lab-on-a-chip devices, genetic analysis devices, cell and/or tissue trapping devices, skin patches, subcutaneous sensors, intramuscular sensors, any and all FDA-regulated medical devices in perpetuity;

“Medication” as used herein is defined to include, but not be limited to, prescription medications, pills, capsules, tablets, time-release containers, oils, lotions, creams, ointments, suppositories, injections, nasal sprays, nasal or oral or vaginal swabs, powders, liquids, drops, inhalants, soaps, shampoos, patches, compounds, intravenous fluids, implants, devices inserted into the human body, energy (e.g., including but not limited to magnetic, kinetic, light, heat, microwave, radio, ultrasound, or sound waves, etc.), beverages, formulas, viruses, engineered immunity, genetic engineering, shunts, 3D-printed synthetics, dietary supplements, vitamins, ultrasound, gas bubbles or bubbles permeating the blood-brain barrier, or any physical, energy, chemical or biological treatment that has a chemical or biological effect on a human.

“Module” as used herein is defined to include, but not be limited to, computer programming logic, systems, methods, functions, or operations used to provide specific functions regardless of language or format or medium (e.g., including but not limited to hardware, firmware, software, applications, artificial intelligence, adaptive learning systems, networks, nanotechnologies, etc.).

“Output” as used herein is defined to include, but not be limited to, letters, words, symbols, numbers, graphs or tables whether electronically on paper or other medium, videos, sounds, pictures, images, simulations, projections, storytelling, gamification, on-screen displays, on-device displays, heads-up displays, or any and all other formats for the purposes of transmitting information to humans or computers or machines;

“Pharmaceutical Manufacturer” as used herein is defined to include, but not limited to, any corporation, group, or individual involved in the manufacturer or sale of Medication;

“Patient” as used herein is defined to include, but not be limited to, any human or animal receiving, registered, or projected to receive medical treatment, or consume Medication;

“Patient Data” as used herein is defined to include, but not be limited to, any and all Patient identifying or contact information (including, but not limited to, name(s), addresses, telephone or fax numbers, email addresses, direct-messaging addresses (e.g., Facebook messenger, WhatsApp, WeChat, Kik, KaoKao, Twitter messenger, GChat, etc.), SMS Text message numbers, social security numbers, Insurance Carrier plan or identity numbers, national or public health service or Payer numbers), identity or contact information for the Patient's family, friends or caregivers, identity and contact information for any and all health-care providers (including but not limited to physicians, pharmacists, nurses, hospital or clinic or nursing or ambulatory care centers or their agents or employees, or anyone licensed by a public or private entity to provide health, physical, mental, or emotional care to Patients), Medication names, dosages, start dates, end dates, refill dates or consumption or use instructions, vaccine data as to which vaccines, dosages, types or related dates, Medication conflicts, ADRs or AEs, or any other data or information to assist in the efficacy of the invention, or Analysis of any combination(s) of the preceding data types;

“Payers” as used herein is defined to include, but not be limited to, medical insurance companies, governmental entities, or National Health Services (e.g., in countries where universal health care is provided for citizens); however, may include any entity or person paying for a patient or cohort of patients' health care, medications, or treatments. Payers could also include life insurance or disability insurance companies.

“Social Data” as used herein is defined to include, but not be limited to, societal information related to a patient, or cohorts of patients, expressly including financial and/or income status, marital status, sexual activity or habits, education, race or ethnicity, alcohol use, drug use, hair color, skin composition or colors, recreational drug use, tobacco use or exposure, physical activity, social connections, on-line activity, depression, mental or emotional state, stress, employment, financial resource or strains, location, family status (e.g., parent, sibling, etc.), community compositional characteristics, exposure to living or work hazards, exposure to stress, exposure to violence, workplace or residential exposures, animal exposures, environmental exposures or circumstances, profession, hobbies, military or deployment status, and any and all combinations or computations or Analysis of these data points (e.g., averages, extrapolations, rates of change, etc.), or Analysis of any combination(s) of the preceding data types;

“Social Media Data” as used herein is defined to include, but not be limited to, data or knowledge or information gleaned from searching, reviewing, or analyzing peoples' blogs or social media activity (including but not limited to sites or services or companies such as Facebook, Instagram, YouTube, Twitter, Google, Yahoo, Tumblr, Pinterest, LinkedIn, Flickr, Foursquare, Q Zone, Sina Weibo, LINE, Tencent weibo, Youku, Tudou, RenRen, Badoo, Orkut, Vkontakte, MySpace, Snapchat, Reddit, Go.com, Imgur.com, Craig's List, Angie's List, Live.com, Squidoo, Stumpbleupon, Dzone, etc.) or their successors, finance or economic activity (including but not limited to sites or services or companies such as PayPal, EBay, Amazon, Baidu, Mint.com, The Currency Converter, Currency, Click2Sell, WePay, Google Wallet, 2Checkout, Authorize.net, Skrill, Intuit, ProPay, Clickbank, Stripe, etc.) or their successors, travel activity (including but not limited to sites or services or companies such as Expedia, Yelp, Travelocity, Kayak, TripAdvisor, TripIt, GateGuru, UrbanSpoon, OpenTable, Panoramo, SunSeeker, SeatGuru, Waze, SitorSquat, iTraveler, Flight Sites, WorldView, Hotels.com, Priceline, GoogleEarth, Trapster, Wi-Fi Finder, etc.) or their successors, gaming activity (including but not limited to sites or services or companies such as Gametrailers.com, Gamerswithjobs.com, RPGamer, IQN, GameSpot, Kotaku, N4G, PCGamer, NeoSeeker, etc.) or their successors, entertainment activity (including but not limited to sites or services or companies such as NetFlix, AppleTV, iTunes, Hulu, Amazon.com, Pandora, Redbox, etc.) or their successors, health and fitness activity (including but not limited to sites or services or companies such as Cody, Hot5Fitness, Pact, Carrot Fit, Human, Nike FitBit, etc.) or their successors, medical application activity (including but not limited to sites or services or companies such as Epocrates, MedScape, MedCalc, Skyscape, Doximity, Up To Date, etc.) or their successors; and, expressly including any and all direct-messaging applications (e.g., Facebook Messenger, WeChat, WhatsApp, Google Chat, KaoKao, Vine, Kik, etc.) or their successors, or Analysis of any combination(s) of the preceding data types;

“Sources” as used herein is defined to include, but not be limited to things capable of being sources for data input including, but not limited to, humans or animals or machines or Medical Devices, Wearable computers, Internet-of-Things Devices, Internet-enabled or Smart Devices, Ingestible Technologies, Electronic Health Records (EHR) or successors, medical information systems, sensors, nanowire devices, nanosensors, microchips, biomolecular computers or devices, biological transducers, satellites, networks, smart blood, dynamic systems, wireless transmitters, optical transmitters, body-part trackers, gesture trackers (e.g., Wii, etc.), movement trackers, smart furniture (e.g., beds, chairs, etc.) that measure or report data, radio-frequency tags (e.g., RFID, etc.), e-skin (whether projected onto the skin as a screen, topical or intradermal), bar codes, sensing systems, active tags, physiological status-monitoring systems or applications or successors, aircraft, watercraft, armor, prosthetics, social robots (e.g., JIBO, etc.), vehicles, drones, autonomous devices, or Apps or Applications.

“Storage” as “Store” used herein is defined to include, but not be limited to, computer memory, primary computer storage devices (e.g., RAM), secondary computer storage devices (e.g., hard drives, etc.), other types of electronic or data storage, optical disks, records in any medium, data hubs, clouds, caches, networks, quantum computers or storage devices, biologically-based storage devices (e.g., DNA storage), silicon-based storage, arrays or groups or collections or combinations of any of the foregoing, or any other device used for recording information;

“Transmission Method” as used herein is defined to be the methods by which data is transmitted to the system or modules to operate, which expressly includes, but is not limited to, network-to-network, Internet or successors, local area networks, wide area networks, wi-fi (e.g., IEEE 802.11x) or successors, Li-Fi, optical wireless communications or successors, radio frequency (RF) communications or successors, cellular networks, virtual private network (VPN) or successors, ISO/IEEE 11073 medical device protocols or successors, Bluetooth or successors, distributed computing systems or successors, data clouds or successors, IPN (InterPlaNet) or its successors, networked nodes or satellites, networks of networks, space communications protocol specification (SCPS) or successors, SCPS-like, delay-tolerant networking (DTN), bundle protocols (BP), CCSDS file delivery protocols or successors, coherent file distribution protocols or successors, SIPRNet or its successors, RN-800 or successors, next-generation enterprise networks (NGEN) or successors, secure operational infrastructure and communication (SONIC) or successors, optical networks or successors, laser-based communication or successors, mobile networks, nanowire devices, nanosensors, microchips, e-skin, OSI physical layers (e.g., including but not limited to Fast Ethernet, RS232, ATM, Ethernet, FDDI, B8ZS, V.35, V.24, RJ45, etc.), OSI data link layers (e.g., including but not limited to Media Access Control (MAC), Logical link control (LLC), PPP, FDDI, ATM, IEEE 802.5/802.2, IEEE 802.3/802.2, HDLC, Frame Relay, etc.), OSI network layers (e.g., including but not limited to AppleTalk DDP, IP, IPX), OSI transport layers (e.g., including but not limited to SPX, TCP, UDP, etc.), OSI session layers (e.g., including but not limited to NFS, NetBios names, RPC, SQL, etc), or networks of smart or any or all of the foregoing, or any combination of some or all of these transmission methods.

“User Interface” as used herein is defined to include, but is not limited to, graphical or gestural or optical or lingual (spoken) or manual interfaces whether kinetic or sensing, whether tangible (e.g., matter) or virtual (e.g., including but not limited to electronic or heads-up displays), keyboards (physical, virtual or electronic), mouse point-and-click systems, command lines, code, other Applications or Modules, touchscreens, OSI presentation layers (e.g., including but not limited to ASCII, EBCDIC, TIFF, GIF, PICT, JPEG, MPEG, etc.), brain-computer interfaces (e.g., including but not limited to EPOC neuroheadset, etc.), flexible OLED displays, augmented reality, voice user interfaces (VUI) (e.g., including but not limited to Siri, etc.), tangible user interfaces (TUI) (e.g., including but not limited to Microsoft Pixelsense, Microsoft Table 1.0, etc.), sensor network user interfaces (SNUI)(e.g., including but not limited to Siftables, etc.) or natural language user interfaces or the successors of any of the above herein.

“Virtual Data” as used herein is defined to include, but is not limited to, elements of or the collective data of users' on-line activities or profiles, active or passive digital footprints, on-line user metadata, Internet-searching (e.g., including but not limited to Google, Bing, etc.), results or patterns or habits, Internet activities (e.g., including but not limited to what sites are visited, how long, how often, or what occurs when users are on that site), social media activities (e.g., including but not limited to Facebook, Vine, etc.), on-line shopping (e.g., including but not limited to Amazon, E-Bay, etc.), on-line mapping (e.g., including but not limited to Google maps, Apple maps, etc.), communication-command-control (3C) systems, trails or traces that are generated by peoples' on-line activities, computer “cookies” or “super-cookies,” or Analysis of any combination(s) of the preceding data types;

“Wearable Computers” as used herein is defined to include, but is not limited to any Internet or Intranet or network-enabled device or device capable of communicating electronically or optically or via radio or other frequencies including, but not limited to, watches (e.g., including but not limited to iWatch, LG Urban, etc.), eyeglasses (e.g., including but not limited to Google glass, etc.), clothing (e-textiles), armor, jewelry (e.g., including but not limited to bracelets, rings, necklaces, etc.), helmets or headgear or visors, masks, visors, gloves, shoes or boots, coats, jackets, vests, exoskeletons (e.g., including but not limited to products such as TALOS, F-INSAS, FORTIS, HULC, those used in “Soldier of the Future” systems, or any successors), body suits (part or whole), belts, ear pieces (internal or external), throat microphones or sensors, headbands, ear muffs or phones or coverings, oral implants, skin implants, scarves, bras and/or undergarments, or armor.

### Additional Configuration Considerations

Some portions of the above or below descriptions describe the embodiments in terms of algorithmic processes or operations. These algorithmic descriptions and representations are commonly used by those skilled in the data processing arts to convey the substance of their work effectively to others skilled in the art. These operations, while described functionally, computationally, or logically, are understood to be implemented by computer programs comprising instructions for execution by a processor or equivalent electrical circuits, microcode, pseudo-code, or the like. Furthermore, it has also proven convenient at times, to refer to these arrangements of functional operations as modules, without loss of generality. The described operations and their associated modules may be embodied in software, firmware, hardware, or any combination thereof. In all cases, the language of this disclosure and coverage of proprietary claims is maximally expansive and non-restrictive, and figurative more than literal.

As used herein, any reference to “one embodiment” or “an embodiment” or means that a particular element, feature, structure, or characteristic described in connection with the embodiment is included in at least one embodiment. The appearance of the phrase “in one embodiment” in various places in the specification are not necessarily all referring to the same embodiment.

As used herein, the terms “compromises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such processes, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the disclosure. The description should be read to include one or at least one and the singular also includes the plural unless it is obvious that it is meant otherwise.

Upon reading this disclosure, those of skill in the art will appreciate still additional alternative structure and functional designs. Thus, while particular embodiments and applications have been illustrated and described, it is to be understood that the described subject matter is not limited to the precise construction and components disclosed herein and the various modifications, changes, and variations that will be apparent to those skilled in the art may be made in the arrangement, operation, and details of the method and apparatus disclosed herein.

The term “Rx&You” is a proprietary name for the collective technologies herein and where and when used refers comprehensively to the entire body of systems and methods and may be used to preference a description or drawing of a specific component, sub-component, module, component, function, element, or application. RxYou is used interchangeably herein to refer to the entire Platform, Application, Software, and collection of modules, code, pseudo-code, algorithms, schemas, triggers, rules, analysis engines, and User Interfaces.

### DIAGRAMS OR DRAWINGS

### Introductory Note:

The diagrams and drawings are approximately organized as follows: 1-4 are network and system architectures and schemas; 5 is a flowchart algorithm of system decisions; 6-10 are data flows; 11-20 are Patient-related user interfaces; 21-24 are Provider-related user interfaces (which could also apply to Payers or Pharmaceutical Manufacturers); 25-28 and 30-31 are flowchart algorithms; 29 is a Big-data as a Service (BDaaS) technology structure; and, 32 is a flowchart algorithm for and description of a rules-based inference engine.

What is claimed is:

1. A computer-implemented method for engaging patients or users via interfaces or devices to maximize medication adherence, persistence, clinical outcomes, education, and safety, and enable real-time analysis, prioritizations, alerts, and reporting; the method comprising:
a. Identifying, patients or users and patient-related or user-related data on a back-end system, which includes but is not limited to names, dosages, prescriptions, durations, prescribers, interactions, and adverse events for medications, details about current and former providers, and genomic data from payers, providers, vendors, or pharmaceutical manufacturers;
b. Importing data from Sources via an application programming interface (API) to structured and/or unstructured databases in batch jobs or in streams, which are hosted on the cloud and/or remote data center(s) and transmitted electronically or wirelessly, including but not limited to via the Internet, file transfer protocols (FTPs), and/or hypertext transfer protocols (HTTP);
c. Identifying, from patient or user interactions on smart devices or a telephone, data, feedback, and behaviors regarding mediation usage, including but not limited to, adherence, persistence, adverse events, interactions, and regarding medication efficacy, including but not limited to how the patients thinks, appears, or feels;
d. Importing data from Sources via one or more application programming interfaces (APIs) to structured and/or unstructured databases in batch jobs or in streams, which may be hosted on the cloud and/or remote data center(s), and transmitted electronically or wirelessly, including but not limited to via the Internet, file transfer protocols (FTPs), and/or hypertext transfer protocols (HTTP).
e. Identifying data from patients' or users' devices and/or sensors (e.g., including but not limited to smart watches, environmental sensors, embedded or consumed sensors, smart clothing, etc.) or data Sources (as defined herein) regarding patients' or users' Data (as defined herein);
f. Importing data from Sources via one or more application programming interfaces (APIs) to structured or unstructured databases in batch jobs or in streams, which may be hosted on the cloud and/or remote data center(s), and transmitted electronically or wirelessly, including but not limited to via the Internet, file transfer protocols (FTPs), and/or hypertext transfer protocols (HTTP);
g. Identifying data from patients' or users' use of computers or User Devices (e.g., including but not limited to social media, Internet searches, purchases, consumer preferences, locations, travel, etc.);
h. Importing data from Sources (as defined herein) described in 1g via one or more application programming interfaces (APIs) to structured or unstructured databases in batch jobs or in streams which may be hosted on the cloud and/or remote data center(s), and transmitted electronically or wirelessly, including but not limited to via the Internet, file transfer protocols (FTP), and/or hypertext transfer protocols (HTTP);
i. Identifying data or Data Sources (as defined herein) regarding genomic, epigenetic, and/or proteinomics sequence and/or variances Data inside or related to patients' or users' bodies from patients, providers, or third-party vendors;
j. Importing data from Data Sources via one or more application programming interfaces (APIs) to structured or unstructured databases in batch jobs or in streams which may be hosted on the cloud and/or remote data center(s), and transmitted electronically or wirelessly, including but not limited to via the Internet, file transfer protocols (FTPO, and/or hypertext transfer protocols (HTTP);
k. Identifying patients' or users' interactions intra-regimen, inter-regimen, intra-treatment, or inter-treatments;
l. Alerting patients, users, providers, payers, pharmaceutical manufacturers, governments, national health services, insurers, and/or public health agencies about interactions via Output(s);
m. Identifying safety risks, including as examples but not limited to interactions, critical dosing, Beers criteria for age-appropriate medications and dosing, etc.;
n. Alerting patients, users, providers, payers, pharmaceutical manufacturers, governments, national health services, insurers, and/or public health agencies about interactions identified in 1m via Output(s);
o. Identifying at-risk patients, regiments, and behaviors using predictive analytics, including as examples but not limited to, the Hogan Drug Attitude Inventor (DAI), Morisky-4, Morisky-8, Medication Adherence Rating Scale (MARS), etc.;
p. Alerting patients, users, providers, payers, pharmaceutical manufacturers, governments, national health services, insurers, and/or public health agencies about interactions via Output(s);
q. Identifying patients' or users' educational needs regarding medication(s), treatment(s), safety, prevention, diagnoses, or prognoses based on historical, present, or future medication regimens or healthcare needs;
r. Digitally delivering micro-targeted content to patients or users to address items via slide decks, streaming videos, streaming audio, or other visual or educational aids;
s. Identifying adverse or unexpected events regarding medication and enabling patients or users to electronically answer questions and report them to providers, payers, insurers, national health services, governments, pharmaceutical manufacturers, or public-health agencies;
t. Enabling patients or users the ability to track and manage adverse events by patient or user identity, type of adverse event, medication(s) involved, effects, frequency, time, and stage of review or remedy cumulatively overtime and patient or user cohorts;
u. Identifying patients' or users' medication types by icons on a user-interface for quality assurance;
v. Identifying all the persons or offices involved in the healthcare of a patient and all their pertinent contact information;
w. Enabling patients, providers, or users to have visibility to which healthcare personnel are treating a patient, how, when, and why, and to receive alerts regarding the treatment, behaviors, events, or outcomes that may have derived from other professionals' treatments;
x. Identifying which time brackets (e.g. meal times) that a patient or user is prescribed to use medications or treatments;
y. Alerting patients or users at said time windows as to what medication treatments or uses they are directed by their healthcare personnel to take or use via Output(s);
z. Enabling two-way communication with patients or users to confirm that they have taken or used the medication or treatment as directed (in response to an alert), have not, or have but in an undirected way);
aa. Enabling multi-cultural patient or user feedback via traffic-light style color coding of green, yellow, and red for acceptable or directed medication or treatment use, quasi-compliance or adherence, and non-adherence, respectively;
bb. Enabling patients or users to edit which time window they receive alerts for medications or treatments, turning off alerts by day of the week, modifying the time of alerts on a 24-hour clock, and modifying the communication method by which they receive alerts on a medication-by-medication—or treatment-by-treatment basis—to include but not be limited to alerts via telephone, email, SMS Text, and direct-messaging platforms (e.g., Skype, Twitter, Facebook Messenger, WeChat, KaoKao, etc.);
cc. Reporting to patients or users an event log of every medication ever taken by time-date stamp, level of compliance (green-yellow-red), and dosage in tabular or various graphical formats;
dd. Enabling patients or users to edit and modify the medications in their regimen(s), and providers or healthcare professionals on their team, including their pertinent information;
ee. Applying artificial intelligence via machine learning, or inference-based rules engine, to conduct analysis, prioritizations, and knowledge discovery related to all data identified herein;
ff. Linking databases for structured data (e.g., SQL, Oracle, etc.) and unstructured data (e.g., MongoDB) databases in such a way that they can receive or ingest any type of data, and analysis, querying, or reporting can occur across the linked databases as if they were one enterprise;
gg. Enabling patients or users to attach efficacy, satisfaction, value or similar quality-control quantifiable scores (e.g., Total Satisfaction Quality Measurements or TSQM) to medications or regimens for comparative effectiveness research;
hh. Analyze by combining data elements (e.g., efficacy scores and genetic variants and medications) new discoveries and research results to maximize clinical care and outcomes, safety, and value;
ii. Enabling digital real-estate on patient or user portals in which advertisements may be streamed or presented to patients in any data format micro-targeted to any data points relative to that patient or user (e.g., location, treatments, medications, etc.);
jj. Identifying patients' or users' pharmacokinetic (PK) curves for customized dosing;
kk. Enabling patients' or users' PK curves in item 1jj to be automatically connected to the alerting, analysis, and reporting functions to ensure patients' or users' can dose within their PK curve, and report and analyze their effectiveness at doing so (e.g., to support pay for patient performance);
ll. Enabling patients' or users' medication or treatment data to be received or ingested from wireless medication and dosing monitors (including those ingested, implanted, and worn on the skin) to allow for remote monitoring, analysis, alerting, and reporting relative to patients' or users' performance, behaviors, and medication needs (e.g., Proteus, Insulet OmniPod, etc.);
mm. Enabling patients or users to store their medication and medical data or information in a cloud-based personal storage locker to which they may grant others access remotely (e.g., to comply with data “shelf-life” laws in Korea and other countries);
nn. Identifying patients' or users' medication or treatment risks, behaviors, events, or opportunities automatically via artificial intelligence; and,
oo. Enabling patients' or users' risks, behaviors, events, or opportunities identified in item 1nn to be responded or reacted to automatically via artificial intelligence advising patients or users on specific courses of action or conduct.

2. A computer-implemented method to engage users via interfaces or devices and/or for applying systems science to population health; the method comprising:
a. Complex event processing (CEP) to ingest Data or streams of Data (as defined herein to include but not be limited to behavioral, biometric, environmental, epidemiological, pharmacovigilant, medical, social, social media, or virtual) from multiple Sources (as defined herein, including but not limited to (IoT, user devices, enterprise platforms or system, etc.);
b. Linked databases to store structured and unstructured data that is ingested;
c. Artificial intelligence (e.g., machine learning, inference-based rules engines, and/or algorithms based on statistics and modeling) to analyze Data or streams of Data from Sources; and,
d. Output (as defined herein) based on the analysis, which may expressly include but not be limited to alerts, reports, notices, graphics, numbers, words, calculations, or predictions via any Transmission Method (as defined herein).

3. The methods according to claim 1, wherein any Data from Data Sources (as defined herein) are applied to operationalize medical information or intelligence in or near real-time such that an application identifies, prioritizes, analyzes, and/or delivers necessary information at an appropriate time to an appropriate person or organization via an appropriate Output to allow said recipients to discover additional or new information that could or improve the well-being of a patient or user.

4. The methods according to claim 1, wherein any Data from Data Sources are applied to coordinates care across or among healthcare providers in disparate locations and/or remotely in such a way that it could help or improve the well-being of a patient or user.

5. The methods according to claim 1, wherein any Data from Data Sources are applied to customizes medical treatment at a personal level in such a way that it could help or improve the well-being of a patient or user.

6. The methods according to claim 1, wherein any Data (as defined herein) from any Data Sources are applied to enables infonomics or the discovery of new information, knowledge, or trends that may benefit humankind at a population health level.

7. The methods according to claim 1, wherein any Data (as defined herein) from any Data Sources (as defined herein) are applied to smart textiles, suits, sensors, uniforms, or armor in such a way as to assist, help, or improve the safety or well-being of soldiers, professional, transportation, manufacturing, or industrial workers, astronauts or space travelers or workers, divers or underwater workers, miners or underground workers, athletes or individuals in competitive or recreational activities.

8. The methods according to claim 1, wherein any Data (as defined herein) from any Data Sources (as defined herein) are applied to digital therapies or the discovery of new information, behaviors, risks, opportunities, knowledge, or trends that may benefit patients or users by allowing them to modify behavior or engage in other non-medication treatments to forego or prevent the consumption of medications.

9. The methods according to claim 1, wherein pharmaceutical manufactures, institutions, people, or organizations, apply the system or methods to clinical trials.

10. The methods according to claim 1, wherein governments, government agencies, or national health services apply the system or methods toward public health goals.

11. The methods according to claim 1, wherein payers, health insurers, and/or workers' compensation insurers apply the system or methods toward cost savings, improved safety, efficacy, or efficiency.

12. The methods according to claim 1, wherein companies, institutions, organizations, or people, apply the system or methods towards health goals.

13. The methods according to claim 1, wherein companies, institutions, organizations, or people, apply the system or methods towards clinical decision support systems (CDS).

14. The methods according to claim 1, wherein governments, companies, institutions, organizations, or people apply the system or methods toward public health goals, efficacy, safety, value, or efficiency.

15. The methods according to claim 1, wherein companies, institutions, organizations, or people, apply the system or methods towards connecting to, with, or from electronic health records (EHRs) or electronic medical records (EMRs).

16. The methods according to claim 1, wherein companies, institutions, organizations, or people, apply the system or methods towards connecting to, with, or from electronic prescribing systems and/or computerized physician or provider order entry (CPOE) systems.

17. The methods according to claim 1, wherein companies, institutions, organizations, or people apply the system or methods towards electronic or digital rights management (DRM).
